(19) United States
(12) Patent Application Publication
(10) Pub. No.: US 2022/0185895 A1
(43) Pub. Date: Jun. 16, 2022

(54) METHODS OF REDUCING LARGE GRANULAR LYMPHOCYTE AND NATURAL KILLER CELL LEVELS

(71) Applicant: Dren Bio, Inc., Redwood City, CA (US)

(72) Inventors: Nenad TOMASEVIC, Foster City, CA (US); Ruo Shi SHI, San Mateo, CA (US); Arun KASHYAP, Newark, CA (US)

(73) Assignee: Dren Bio, Inc., Redwood City, CA (US)

(21) Appl. No.: 17/599,481

(22) PCT Filed: Mar. 26, 2020
(86) PCT No.: PCT/US2020/025012

§ 371 Date: Sep. 28, 2021

Related U.S. Application Data
(60) Provisional application No. 62/982,578, filed on Feb. 27, 2020.
(60) Provisional application No. 62/826,660, filed on Mar. 29, 2019.

(51) Int. Cl. C07K 16/28

(52) U.S. Cl. CPC C07K 16/2851; C07K 16/2896; C07K 2317/92; C07K 2317/732; C07K 2317/21; C07K 16/2803

(57) ABSTRACT

The present disclosure relates to methods of treating diseases or disorders associated with LGL and/or NK cells, methods of reducing or depleting LGL and/or NK cells, and methods of inducing ADCC activity using antibodies that bind to a cell surface protein on LGL and/or NK cells and comprise enhanced ADCC activity. The present invention also relates to a method of depleting or reducing the numbers of large granular lymphocytes and natural killer cells in a human subject upon administration of CD94 or CD57 or NKG2A binding molecule that consists of a part that specifically binds to the CD94 or CD57 or NKG2A receptors and an immunoglobulin Fc part. In a specific embodiment, a method of the invention depletes or reduces the number of large granular lymphocytes and natural killer cells in spleen, blood, bone marrow, joints, or other tissues.

### CROSS REFERENCE TO RELATED APPLICATIONS

This application claims priority to U.S. Provisional Application Nos. 62/826,660, filed Mar. 29, 2019, and 62/982,578, filed Feb. 27, 2020, the disclosures of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 186542000140SEQLIST.TXT, date recorded: Mar. 25, 2020, size: 17 KB).

### FIELD OF THE INVENTION

The present disclosure relates to methods of reducing large granular lymphocyte and natural killer cell levels in humans

### BACKGROUND OF THE INVENTION

Lymphocytes are a subset of white blood cells which specifically recognize and respond to foreign antigen. There are 3 major classes of lymphocytes: T lymphocytes (T cells), B lymphocytes (B cells) and natural killer (NK) cells. Large granular lymphocytes (LGLs) account for 8-15% of the peripheral blood lymphocytes (200-400/μL) and are characterized by abundant cytoplasm with azurophilic granules (Loughran T P Jr. Blood. 1993; 82(1):1-14). The azurophilic granules contain cytolytic components such as perforin and granzymes. The LGLs are divided into two major categories: cytotoxic T- and NK-cells. The LGL T-cells typically express CD3, CD8, and CD57 and show TCR gene rearrangement; whereas the NK-cells express CD56, are negative for surface CD3, may express CD8, and do not show TCR gene rearrangement (Alekshun et al., Cancer Control 2007, Vol. 14, No. 2, p 141-150). NK-cell LGLs (CD3−) belong to the innate immune system and have the capability of non-major histocompatibility complex-restricted cytotoxicity (Alekshun et al., Cancer Control 2007, Vol. 14, No. 2, p 141-150).

There are 3 distinct diseases involving LGLs: T-cell LGL (T-LGL) leukemia; chronic lymphoproliferative disorders of NK cells (CLPD-NK, formerly NK-LGL); and aggressive NK-cell leukemias, such as aggressive natural killer leukemia (ANKL) and extranodal NKL nasal type (ENKL). T-cell LGL leukemia is the most frequent LGL disorder in Western countries and accounts for 85% of all cases. The median age at diagnosis is 60 years, without gender predilection. Pathogenesis of the disease is dominated by a clonal expansion of LGL resistant to activation-induced cell death due to constitutive survival signaling (Lamy et al., Blood, 2017, Vol. 129, No. 9, 1082-1094). About one third of T and NK LGL leukemia patients are asymptomatic at the time of diagnosis. Initial presentation is mainly related to neutropenia and includes recurrent oral aphthous ulcerations, fever secondary to bacterial infections. These infections typically involve skin, oropharynx, and perirectal areas, but severe sepsis may occur. However, some patients may have profound and persistent neutropenia without any infections over a very long period of time. The frequency of recurrent infections varies in different series, from 15% to 39%. Fatigue and B symptoms are observed in 20% to 30% of cases. Splenomegaly is reported with a frequency varying from 20% to 50% and lymphadenopathy is rare. Half of the patients present with lymphocyte counts between 4×10⁹/L and 10×10⁹/L, and the LGL count usually ranges from 1 to 6×10⁹/L. A lower LGL count (0.5 to 1×10⁹/L) may be observed in 7% to 36% of cases. Severe neutropenia and moderate neutropenia are observed in 16% to 48% and 48% to 80% of cases, respectively. Anemia is frequent; transfusion-dependent patients are observed in 10% to 30% of cases (Lamy 2017).

The vast majority of LGL leukemia patients will eventually need treatment at some point during disease evolution. Disease-related deaths are mainly due to severe infections that occur in 10% of the patient population. Overall survival at 10 years is 70% (Lamy 2017). First-line therapies rely on the use of single immunosuppressive oral agents such as methotrexate (10 mg/m2 per week), cyclophosphamide (100 mg per day), or cyclosporine (3 mg/kg per day). Based on retrospective studies, the overall response rate (ORR) median 50% with similar responses to each of the 3 drugs. The complete response (CR) rate is relatively low: 21% for methotrexate, 33% for cyclophosphamide, and 5% for cyclosporine. Duration of response is 21 months for methotrexate and the relapse rate is high, i.e. 67%.

Hepatic (hepatitis) and lung dysfunction (hypersensitivity pneumonitis) may occur upon chronic methotrexate treatment. It is recommended to stop cyclophosphamide administration after 8 to 12 months because of its mutagenic potential. Renal function and blood pressure have to be carefully monitored during cyclosporine treatment.

In addition to the NK or T LGL leukemias, NK or LGL cells play key roles in rheumatoid arthritis (RA), Felty's syndrome, aggressive NK leukemia, Inclusion body myositis (IBM), inflammatory bowel disease (IBD), and other diseases. Felty's syndrome (FS) is characterized by the triad of seropositive rheumatoid arthritis (RA) with destructive joint involvement, splenomegaly and neutropenia. The complete triad is not an absolute requirement, but persistent neutropenia with an absolute neutrophil count (ANC) generally less than 1500/mm³ is necessary for establishing the diagnosis. Approximately 30-40% of FS patients have peripheral blood expansions of LGL. Clonal T-LGL populations are very similar in FS and T-LGLL, with expression of CD3+, CD28−, CD57+ and expression of inhibitory and activating NK receptors on LGL. Symptoms and management of LGLL and SF are similar.

Unlike in synovial fluids (SF) from normal human subjects that have no LGL or NK cells, SF from RA patients have high level of LGL and NK cells expressing CD94 or CD57 or NKG2A. It has been shown that CD94 may be a key regulator of synovial NK-cell cytokine synthesis.

IBM is the most common inflammatory muscle disease in older adults. The disease is characterized by slowly progressive weakness and wasting of both distal and proximal muscles, most apparent in the finger flexors and knee extensors. Inflammation is evident from the invasion of muscle fibers by immune cells. Large granular lymphocyte expansions are present in both blood and muscle and provides additional biomarkers for IBM and suggests a mechanistic relationship to the neoplastic disease T-cell large granular lymphocytic leukemia. Most (58%) patients with IBM have aberrant populations of large granular lymphocytes in their blood meeting standard diagnostic criteria for T-cell LGLL. Muscle immunohistochemistry analysis has demonstrated invasion of large granular lymphocytes into muscle in 15/15 IBM patients but in only 1/28 patients with dermatomyositis or polymyositis.

Current therapies for diseases involving large granular lymphocytes (LGLs) do not selectively reduce the levels of or deplete LGL or NK cells. Accordingly, it would be beneficial to develop more efficacious and safer therapies for treating diseases mediated by LGL and NK cells.

### SUMMARY OF THE DISCLOSURE

The present invention relates to a method of depleting or reducing the numbers of large granular lymphocytes and natural killer cells in a human subject upon administration of CD94 or CD57 or NKG2A binding molecule that consists of a part that specifically binds to the CD94 or CD57 or NKG2A receptors and an immunoglobulin Fc part.

The invention provides a method of treating for treating LGL leukemia, Felty's syndrome, rheumatoid arthritis, aggressive NK leukemia, IBM, or IBD in a subject, comprising administering to said subject an effective amount of an antibody that specifically binds to human CD94, human CD57 or human NKG2A, wherein the antibody comprises a human immunoglobulin Fc region having enhanced ADCC activity as compared to wild type IgG1 Fc region.

The invention provides a method of reducing the number or depleting of peripheral blood LGL or NK cells in a human subject by administering to said subject between about 0.01 to about 25 mg/kg of antibody that is specific for either CD94 or CD57or NKG2A or an additional cell surface protein that is specific for LGL cells and comprising an immunoglobulin Fc region including no fucose or Fc mutations that enhance its binding to CD16 in which the administration of the antibody reduces the number of peripheral blood LGL or NK cells below the limit of detection and the level remains below detection for at least about 1 week after dosing of the antibody. In some embodiments, the reduction of LGL or NK cells takes place within the first 24 hours after administration. In some embodiments, the reduction of LGL or NK cells is reversible. In some embodiments, the reduction in LGL or NK cells leads to a reduction in LGL leukemia symptoms. In some embodiments, the reduction in LGL or NK cells leads to a reduction in Felty's syndrome symptoms. In some embodiments, the reduction in LGL or NK cells leads to a reduction in IBM symptoms. In some embodiments, the reduction in LGL or NK cells leads to a reduction in aggressive NK leukemia symptoms.

In one aspect, provided herein is a method for treating LGL leukemia, Felty's syndrome, rheumatoid arthritis, aggressive NK leukemia, IBM, or IBD in a subject, comprising administering to said subject an effective amount of an antibody that specifically binds to human CD94, human CD57 or human NKG2A, wherein the antibody comprises a human immunoglobulin Fc region having enhanced ADCC activity as compared to wild type IgG1 Fc region. In some embodiments, the administration of the antibody reduces the number of peripheral blood LGL or NK cells below the limit of detection and/or the level remains below detection for at least about 1 week after dosing of the antibody. In some embodiments, the reduction of LGL or NK cells takes place within the first 24 hours after administration. In some embodiments, the reduction of LGL or NK cells is reversible. In some embodiments, said reduction in LGL or NK cells leads to a reduction in LGL leukemia symptoms. In some embodiments, said reduction in LGL or NK cells leads to a reduction in Felty's syndrome symptoms. In some embodiments, said reduction in LGL or NK cells leads to a reduction in IBM symptoms. In some embodiments, said reduction in LGL or NK cells leads to a reduction in aggressive NK leukemia symptoms. In some embodiments, the subject is a mammal In some embodiments, the subject is a human. In some embodiments, the Fc region of the antibody comprises a human IgG1 Fc which is non-fucosylated.

In another aspect, provided herein is a method for treating a disease or disorder in a subject, comprising administering to the subject an effective amount of an antibody that specifically binds to a cell surface protein selected from human CD94, human CD57, or human NKG2A, wherein the antibody comprises a human immunoglobulin Fc region comprising enhanced ADCC activity compared to a wild type IgG1 Fc region, and wherein the disease or disorder is selected from chronic lymphoproliferative disorder of NK cells (CLPD-NK), LGL leukemia, Felty's syndrome, rheumatoid arthritis, aggressive NK leukemia, inclusion body myositis, or inflammatory bowel disease. In some embodiments, administration of the antibody results in a reduction in the number of peripheral blood LGL or NK cells in the subject.

In another aspect, provided herein is a method for reducing the number of peripheral blood LGL and/or NK cells in a subject, comprising administering to the subject an effective amount of an antibody that specifically binds to a cell surface protein selected from human CD94, human CD57, or human NKG2A, wherein the antibody comprises a human immunoglobulin Fc region comprising enhanced ADCC activity compared to a wild type IgG1 Fc region, and wherein the subject has a disease or disorder selected from LGL leukemia, Felty's syndrome, rheumatoid arthritis, aggressive NK leukemia, inclusion body myositis, or inflammatory bowel disease.

In another aspect, provided herein is a method for inducing ADCC activity in a subject, comprising administering to the subject an effective amount of an antibody that specifically binds to a cell surface protein selected from human CD94, human CD57, or human NKG2A, wherein the antibody comprises a human immunoglobulin Fc region comprising enhanced ADCC activity compared to a wild type IgG1 Fc region, wherein the subject has a disease or disorder selected from chronic lymphoproliferative disorder of NK cells (CLPD-NK), LGL leukemia, Felty's syndrome, rheumatoid arthritis, aggressive NK leukemia, inclusion body myositis, or inflammatory bowel disease, and wherein administration of the antibody to the subject results in a reduction in the number of peripheral blood LGL and/or NK cells in the subject.

In some embodiments, which may be combined with any of the preceding embodiments, at least about 1,000 receptors per cell, at least about 2,000 receptors per cell, at least about 3,000 receptors per cell, at least about 4,000 receptors per cell, at least about 5,000 receptors per cell, or at least about 7,000 receptors per cell of the cell surface protein are expressed on the surface of the peripheral blood LGL and/or NK cells in the subject.

In some embodiments, which may be combined with any of the preceding embodiments, the reduction in the number of peripheral blood LGL or NK cells in the subject comprises a reduction of at least about 10%, at least about 20%, at least about 25%, at least about 30%, at least about 40%, at least about 50%, at least about 60%, at least about 70%, at least about 80%, or about 10% to about 80% compared to the number of peripheral blood NK cells in the human subject prior to administration of the antibody. In some embodiments, the reduction in the number of peripheral blood LGL and/or NK cells in the subject occurs within the first 24 hours after administration of the antibody to the subject. In some embodiments, the number of peripheral blood LGL and/or NK cells in the subject is reduced to below the limit for clinical diagnosis of the disease or disorder. In some embodiments, the number of peripheral blood LGL and/or NK cells in the subject is reduced to less than or equal to 2×10⁹ cells/L (e.g., in a peripheral blood sample obtained from the subject). In some embodiments, the reduction in the number of peripheral blood LGL and/or NK cells in the subject to below the limit for clinical diagnosis of the disease or disorder is present in the subject for at least about 1 week after administration of the antibody to the subject. In some embodiments, the reduction in the number of peripheral blood LGL and/or NK cells in the subject to less than or equal to 2×10⁹ cells/L (e.g., in a peripheral blood sample obtained from the subject) for at least about 1 week after administration of the antibody to the subject. In some embodiments, the number of peripheral blood LGL and/or NK cells in the subject is reduced to below the limit of detection for the peripheral blood LGL and/or NK cells in the subject. In some embodiments, the reduction in the number of peripheral blood LGL and/or NK cells in the subject to below the limit of detection for the peripheral blood LGL and/or NK cells is present in the subject for at least about 1 week after administration of the antibody to the subject. In some embodiments, the reduction in the number of peripheral blood LGL and/or NK cells in the subject is reversible.

In some embodiments, which may be combined with any of the preceding embodiments, administration of the antibody to the subject results in a reduction in the number of peripheral blood NK cells in the subject. In some embodiments, the NK cells in the subject are CD3 negative and CD56 positive, CD3 negative and CD16 positive, CD3 negative and CD57 positive, CD3 negative and CD94 positive, or CD3 negative and NKG2A positive. In some embodiments, the antibody has an EC50 of between about 3 ng/ml and about 40 ng/ml.

In some embodiments, which may be combined with any of the preceding embodiments, administration of the antibody to the subject does not result in a reduction of T cells in the subject. In some embodiments, the T cells in the subject are CD3 positive and CD4 positive or CD3 positive and CD16 negative.

In some embodiments, which may be combined with any of the preceding embodiments, the subject is a human

In some embodiments, which may be combined with any of the preceding embodiments, administration of the antibody to the subject does not result in tumor lysis syndrome in the subject.

In some embodiments, which may be combined with any of the preceding embodiments, the antibody comprises a human IgG1 Fc region that is non-fucosylated.

In some embodiments, which may be combined with any of the preceding embodiments, the antibody binds to a human cellular Fc gamma receptor IIIA to a greater extent than an antibody comprising a wild type human IgG1 Fc region. In some embodiments, the human cellular Fc gamma receptor MA comprises a valine residue or a phenylalanine residue at amino acid residue position 158. In some embodiments, the human cellular Fc gamma receptor IIIA comprises the sequence of SEQ ID NO: 8 or 9.

In some embodiments, which may be combined with any of the preceding embodiments, the antibody: (a) specifically binds to human CD94, wherein the antibody does not bind to the same epitope on human CD94 as anti-CD94 antibody clones HP-3D9, DX22, 131412, or 12K45; (b) specifically binds to human CD57, wherein the antibody does not bind to the same epitope on human CD57 as anti-CD57 antibody clone NK-1; or (c) specifically binds to human NKG2A, wherein the antibody does not bind to the same epitope on human NKG2A as anti-NKG2A antibody clone Z199.

In some embodiments, which may be combined with any of the preceding embodiments, the antibody: (a) specifically binds to human CD94, wherein the antibody binds to human CD94 with a greater affinity than anti-CD94 antibody clones HP-3D9, DX22, 131412, and 12K45; (b) specifically binds to human CD57, wherein the antibody binds to human CD57 with greater affinity than anti-CD57 antibody clone NK-1; or (c) specifically binds to human NKG2A, wherein the antibody binds to human NKG2A with a greater affinity than anti-NKG2A antibody clone Z199.

In some embodiments, the disease or disorder is Felty's syndrome, wherein administration of the antibody to the subject results in a reduction of one or more Felty's syndrome symptoms in the subject.

In some embodiments, the disease or disorder is inclusion body myositis, wherein administration of the antibody to the subject results in a reduction of one or more inclusion body myositis symptoms in the subject.

In some embodiments, the disease or disorder is aggressive NK leukemia, wherein administration of the antibody to the subject results in a reduction of one or more aggressive NK leukemia symptoms in the subject.

In some embodiments, the disease or disorder is rheumatoid arthritis, wherein administration of the antibody to the subject results in a reduction of one or more rheumatoid arthritis symptoms in the subject.

In some embodiments, the disease or disorder is LGL leukemia, wherein administration of the antibody to the subject results in a reduction of one or more LGL leukemia symptoms in the subject.

In some embodiments, the disease or disorder is CLPD-NK, wherein administration of the antibody to the subject results in a reduction of one or more CLPD-NK symptoms in the subject.

In another aspect, provided herein is a method for treating CLPD-NK in a human subject in need thereof, comprising administering to the human subject an effective amount of an antibody, wherein the antibody specifically binds to human NKG2A, and wherein the antibody comprises a human immunoglobulin Fc region comprising enhanced ADCC activity compared to a wild type IgG1 Fc region. In some embodiments, the antibody does not bind to the same epitope on human NKG2A as anti-NKG2A antibody clone Z199. In some embodiments, the antibody binds to human NKG2A with a greater affinity than anti-NKG2A antibody clone Z199.

In another aspect, provided herein is a method for treating CLPD-NK in a human subject in need thereof, comprising administering to the human subject an effective amount of an antibody, wherein the antibody specifically binds to human CD94, and wherein the antibody comprises a human immunoglobulin Fc region comprising enhanced ADCC activity compared to a wild type IgG1 Fc region. In some embodiments, the antibody does not bind to the same epitope on human CD94 as anti-CD94 antibody clones HP-3D9, DX22, 131412, or 12K45. In some embodiments, the antibody binds to human CD94 with a greater affinity than anti-CD94 antibody clones HP-3D9, DX22, 131412, and 12K45.

In some embodiments, which may be combined with any of the preceding embodiments, administration of the antibody to the human subject results in a reduction in the number of peripheral blood LGL or NK cells in the human subject of at least about 10%, at least about 20%, at least about 30%, at least about 40%, at least about 50%, at least about 60%, at least about 70%, at least about 80%, at least about 90%, or about 10% to about 90%, compared to the number of peripheral blood NK cells in the human subject prior to administration of the antibody. In some embodiments, the NK cells in the human subject are CD3 negative and CD56 positive, CD3 negative and CD16 positive, CD3 negative and CD57 positive, CD3 negative and CD94 positive, or CD3 negative and NKG2A positive. In some embodiments, administration of the antibody to the human subject does not result in a reduction of T cells in the human. In some embodiments, the T cells in the human subject are CD3 positive and CD4 positive or CD3 positive and CD16 negative. In some embodiments, administration of the antibody to the human subject does not result in tumor lysis syndrome in the human In some embodiments, the antibody comprises a human IgG1 Fc region that is non-fucosylated. In some embodiments, the antibody binds to a human cellular Fc gamma receptor MA to a greater extent than an antibody comprising a wild type human IgG1 Fc region. In some embodiments, the human cellular Fc gamma receptor MA comprises a valine residue or a phenylalanine residue at amino acid residue position 158. In some embodiments, the human cellular Fc gamma receptor MA comprises the sequence of SEQ ID NO: 8 or 9. In some embodiments, administration of the antibody to the human subject results in an improvement of one or more CLPD-NK symptoms in the human

All references cited herein, including patent applications and publications, are incorporated by reference in their entirety.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B show the levels of CD94 receptors on immune cells obtained from healthy donors. FIG. 1A shows flow cytometry analysis of CD94 receptor number in a representative healthy donor peripheral blood leukocyte (PBL) sample. Single and live monocyte, granulocyte and lymphocyte populations were gated to identify the indicated immune cell populations. CD94 expression was determined by comparing to fluorescence minus one (FMO) and isotype control antibody. The percent CD94-positive cells is indicated by the double-headed arrow; the CD94 receptor number is provided below each histogram. The dashed arrow indicates that fluorescence corresponding to the APC-conjugated anti-CD94 antibody HP-3D9 increases along the x-axis of the histograms from left to right. FIG. 1B shows the average number of CD94 receptors per cell in the indicated cell types from peripheral blood mononuclear (PBMC) and PBL samples from healthy donors. CD94 expression (using mAb clone HP-3D9) was assessed in six healthy donor PBMC samples; CD94 expression on granulocytes was assessed in PBL samples from 2 donors. Single and live monocyte, granulocyte and lymphocyte populations were gated to identify the indicated immune cell populations. CD94 expression was determined by comparing to fluorescence minus one (FMO) and isotype control antibody. The antibody binding capacity was calculated by fitting a standard curve using APC-labeled molecules of equivalent soluble fluorochrome (MESF) beads. The values shown above each bar represent the average number of CD94 receptors per cell. In FIGS. 1A-1B, Ab =antibody; “Neg” or “negative” indicates that the target was below the threshold for detection (2K receptors per cell); the K notation refers to a multiple of 1000 (e.g., 0.4K=400; 4K=4000; 40K=40,000; and 400K=400,000).

FIGS. 2A-2B show the levels of CD94 receptors on immune cells obtained from T-LGLL patients. FIG. 2A shows flow cytometry analysis of CD94 receptor number in a sample obtained from a CD94^{bright} T-LGLL patient. FIG. 2B shows flow cytometry analysis of CD94 receptor number in a sample obtained from a CD94^{dim} T-LGLL patient. In this case, CD94^{bright} was ˜170K receptors (e.g., threshold was greater than 50K receptors), whereas CD94^{dim} was ˜12K receptors (e.g., threshold was less than 15K receptors). In FIGS. 2A-2B, single and live monocyte and lymphocyte populations along with selective markers were gated to identify the indicated immune cell populations. CD94 expression was determined by comparing to fluorescence minus one (FMO) and isotype control antibody. CD3+CD16+ leukemic cells represented >55% of lymphocytes in this patient PBMC sample, compared to <10% in normal PBMCs in both CD94^{bright} and CD94^{dim} patient samples. The percent CD94-positive cells is indicated by double-headed arrows; the CD94 receptor number is provided below each histogram. The dashed arrow indicates that fluorescence corresponding to the APC-conjugated anti-CD94 antibody HP-3D9 increases along the x-axis of the histograms from left to right. Ab=antibody; “negative” indicates that no CD94 expression was detected; the K notation refers to a multiple of 1000 (e.g., 0.4K=400; 4K=4000; 40K=40,000; and 400K=400,000).

FIG. 3 shows flow cytometry analysis of the levels of CD94 receptors on immune cells obtained from a chronic lymphoproliferative disorder of NK cells (CLPD-NK) patient. Single and live monocyte and lymphocyte populations along with selective markers were gated to identify the indicated immune cell populations. CD94 expression was determined by comparing to fluorescence minus one (FMO) and isotype control antibody. CD3-CD16+ leukemic cells represented 70% of lymphocytes in this patient PBMC sample, compared to 5-10% in normal PBMCs. The percent of CD94-positive cells is indicated by double-headed arrows; the CD94 receptor number is provided below each histogram. The dashed arrow indicates that fluorescence corresponding to the APC-conjugated anti-CD94 antibody HP-3D9 increases along the x-axis of the histograms from left to right. Ab =antibody; “negative” indicates that no CD94 expression was detected; the K notation refers to a multiple of 1000 (e.g., 0.4K=400; 4K=4000; 40K=40,000; and 400K=400,000).

FIGS. 4A-4B show the levels of NKG2A receptors on NK cells obtained from a healthy donor, as well an ADCC assay of NK cells in PBMCs from a healthy donor. FIG. 4A shows flow cytometry analysis of NKG2A receptor number on CD3-CD56^{bright} NK cells obtained from a healthy donor. Single and live lymphocytes were gated to identify CD3-CD56+NK cells. CD3-CD56^{bright} NK cells were selected because 100% of this cell population expressed NKG2A. NKG2A expression was determined by comparing to fluorescence minus one (FMO) and isotype control antibody. The NKG2A receptor number is provided below the histogram. The dashed arrow indicates that fluorescence corresponding to the APC-conjugated anti-NKG2A Z199 antibody increases along the x-axis of the histogram from left to right. Ab =antibody; the K notation refers to a multiple of 1000 (e.g., 0.4K=400; 4K=4000; 40K=40,000; and 400K=400,000). FIG. 4B shows an ADCC assay using PBMCs from a healthy donor. PBMCs were incubated overnight with the indicated concentrations of isotype control antibody, anti-NKG2A Z199 fucosylated antibody, or anti-NKG2A Z199 non-fucosylated antibody. The percent CD3-CD56^{bright} NK cells remaining was calculated by normalizing to the number of NK cells in the isotype control wells. The EC50 of each antibody was calculated in Graphpad Prism. a-Fuco=non-fucosylated; Fuco=fucosylated; Z199=anti-NKG2A antibody Z199.

FIGS. 5A-5B show the levels of NKG2A receptors on T cells obtained from a healthy donor, as well an ADCC assay of T cells in PBMCs from a healthy donor. FIG. 5A shows flow cytometry analysis of NKG2A in single and live lymphocytes gated to identify CD3+CD8+ T cells. NKG2A expression was determined by comparing to fluorescence minus one (FMO) and isotype control antibody. The percent NKG2A-positive cells is indicated by the double-headed arrow; the NKG2A receptor number is provided below the histogram. Fluorescence corresponding to the APC-conjugated anti-NKG2A Z199 antibody increases along the x-axis of the histogram from left to right. Ab =antibody; the K notation refers to a multiple of 1000 (e.g., 0.4K=400; 4K=4000; 40K=40,000; and 400K=400,000). FIG. 5B shows an ADCC assay of CD3+CD8+ T cells in a healthy donor PBMC sample. Cells were treated with the indicated concentrations of isotype control antibody, anti-NKG2A Z199 fucosylated antibody, or anti-NKG2A Z199 non-fucosylated antibody overnight. The percent T cells remaining was calculated by normalizing to the number of T cells in the isotype control wells. a-Fuco=non-fucosylated; Fuco=fucosylated; Z199=anti-NKG2A antibody Z199.

FIGS. 6A-6B show the levels of NKG2A receptors on NK cells obtained from a patient with CLPD-NK, as well an ADCC assay in CLPD-NK patient-derived PBMCs. FIG. 6A shows flow cytometry analysis of NKG2A receptor number on single and live lymphocytes gated to identify CD3-CD16+ NK leukemic cells. NKG2A expression was determined by comparing to fluorescence minus one (FMO) and isotype control antibody. The NKG2A receptor number is provided below the histogram. The dashed arrow indicates that fluorescence corresponding to the APC-conjugated anti-NKG2A Z199 antibody increases along the x-axis of the histogram from left to right. Ab=antibody; the K notation refers to a multiple of 1000 (e.g., 0.4K=400; 4K=4000; 40K=40,000; and 400K=400,000). FIG. 6B shows an ADCC assay using PBMCs from a patient with CLPD-NK. PBMCs were incubated overnight with the indicated concentrations of isotype control antibody or anti-NKG2A Z199 non-fucosylated antibody. The percent leukemic cells remaining was calculated by normalizing to the number of leukemic cells in the isotype control wells. The EC50 was calculated in Graphpad Prism. Z199 a-Fuco=anti-NKG2A Z199 non-fucosylated antibody.

FIGS. 7A-7B show the levels of NKG2A receptors on normal T cells obtained from a patient with CLPD-NK, as well an ADCC assay on normal T cells from a CLPD-NK patient. FIG. 7A shows flow cytometry analysis of NKG2A in single and live lymphocytes gated to identify CD3+CD16− T cells. NKG2A expression was determined by comparing to fluorescence minus one (FMO) and isotype control antibody. The NKG2A receptor number is provided below the histogram. The dashed arrow indicates that fluorescence corresponding to the APC-conjugated anti-NKG2A Z199 antibody increases along the x-axis of the histogram from left to right. Ab=antibody; “negative” indicates that no CD94 expression was detected; the K notation refers to a multiple of 1000 (e.g., 0.4K=400; 4K=4000; 40K=40,000; and 400K=400,000). FIG. 7B shows an ADCC assay of CD3+CD16− T cells from a CLPD-NK patient PBMC sample. Cells were treated with the indicated concentrations of isotype control antibody or anti-NKG2A Z199 non-fucosylated antibody overnight. The percent T cells remaining was calculated by normalizing to the number of T cells in the isotype control wells. Z199 a-Fuco=anti-NKG2A Z199 non-fucosylated antibody.

FIGS. 8A-8B show the levels of CD94 and NKG2A receptors in representative normal liver tissue. Single and live liver-derived CD45− cells and lymphocyte populations (CD45/CD4/CD8/CD19/CD56+) were examined to screen for CD94 and NKG2A expression. Receptor expression was determined by comparing to fluorescence minus one (FMO) and isotype control antibody. The receptor number in CD94 and NKG2A positive cells is provided below each histogram; the percentages of receptor-positive cells are indicated by double-headed arrows. The dashed arrows indicate that fluorescence corresponding to the APC-conjugated anti-CD94 antibody HP-3D9 (FIG. 8A) or APC-conjugated anti-NKG2A Z199 antibody (FIG. 8B) increases along the x-axis of the histograms from left to right. Ab=antibody; the K notation refers to a multiple of 1000 (e.g., 0.4K=400; 4K=4000; 40K=40,000; and 400K=400,000). “Negative” indicates that target expression was not detected.

FIGS. 9A & 9B show the results of an antibody-dependent cellular cytotoxicity (ADCC) assay in T-LGLL patient PBMCs using the anti-NKG2A antibody Z199 (FIG. 9A) or isotype control (FIG. 9B).

FIG. 10 shows the expression of CD94 over time in normal NK cells cultured with IL-2.

### DETAILED DESCRIPTION OF THE INVENTION

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “comprising” as used herein is synonymous with “including” or “containing”, and is inclusive or open-ended.

Any reference to “or” herein is intended to encompass “and/or” unless otherwise stated. As used herein, the term “about” with reference to a number refers to that number plus or minus 10% of that number. The term “about” with reference to a range refers to that range minus 10% of its lowest value and plus 10% of its greatest value.

### I. Uses and Methods of Treatment

As discussed above, LGL and NK cells have been implicated in the pathogenesis of numerous diseases and disorders. Many of these disorders or diseases are characterized by an accumulation of clonal or non-clonal LGL and NK cells.

In some embodiments, provided herein is a method for treating a disease or disorder in a subject, comprising administering to the subject an effective amount of an antibody that specifically binds to a cell surface protein selected from human CD94, human CD57, or human NKG2A, wherein the antibody comprises a human immunoglobulin Fc region comprising enhanced ADCC activity compared to a wild type IgG1 Fc region, and wherein the disease or disorder is selected from chronic lymphoproliferative disorder of NK cells (CLPD-NK), LGL leukemia, Felty's syndrome, rheumatoid arthritis, aggressive NK leukemia, inclusion body myositis, or inflammatory bowel disease.

In some embodiments, administration of the antibody results in a reduction in the number of peripheral blood LGL and/or NK cells in the subject. In some embodiments, administration of the antibody results in a reduction in the number of peripheral blood LGL cells in the subject. In some embodiments, administration of the antibody results in a reduction in the number of peripheral blood NK cells in the subject.

Also provided herein is a method for reducing the number of peripheral blood LGL and/or NK cells in a subject, comprising administering to the subject an effective amount of an antibody that specifically binds to a cell surface protein selected from human CD94, human CD57, or human NKG2A, wherein the antibody comprises a human immunoglobulin Fc region comprising enhanced ADCC activity compared to a wild type IgG1 Fc region, and wherein the subject has a disease or disorder selected from LGL leukemia, Felty's syndrome, rheumatoid arthritis, aggressive NK leukemia, inclusion body myositis, or inflammatory bowel disease.

Also provided herein is a method for inducing ADCC activity in a subject, comprising administering to the subject an effective amount of an antibody that specifically binds to a cell surface protein selected from human CD94, human CD57, or human NKG2A, wherein the antibody comprises a human immunoglobulin Fc region comprising enhanced ADCC activity compared to a wild type IgG1 Fc region, wherein the subject has a disease or disorder selected from chronic lymphoproliferative disorder of NK cells (CLPD-NK), LGL leukemia, Felty's syndrome, rheumatoid arthritis, aggressive NK leukemia, inclusion body myositis, or inflammatory bowel disease, and wherein administration of the antibody to the subject results in a reduction in the number of peripheral blood LGL and/or NK cells in the subject.

In some embodiments, the antibody specifically binds to human CD94 or human NKG2A. In some embodiments, the antibody specifically binds to human CD94. In some embodiments, the antibody specifically binds to human NKG2A. In some embodiments, the disease or disorder is CLPD-NK. In some embodiments, the disease or disorder is LGL leukemia. In some embodiments, the disease or disorder is Felty's syndrome. In some embodiments, the disease or disorder is rheumatoid arthritis. In some embodiments, the disease or disorder is aggressive NK leukemia. In some embodiments, the disease or disorder is inclusion body myosistis. In some embodiments, the disease or disorder is inflammatory bowel disease. In some embodiments, the disease or disorder is T-large granular lymphocyte leukemia (T-LGLL). In some embodiments, the disease or disorder is Natural Killer-large granular lymphocyte leukemia (NK-LGLL). In some embodiments, the disease or disorder is CLPD-NK and the antibody specifically binds to human CD94. In some embodiments, the disease or disorder is CLPD-NK and the antibody specifically binds to human NKG2A. In some embodiments, the disease or disorder is T-LGLL and the antibody specifically binds to human CD94. In some embodiments, the disease or disorder is T-LGLL and the antibody specifically binds to human NKG2A. In some embodiments, the disease or disorder is NK-LGLL and the antibody specifically binds to human CD94. In some embodiments, the disease or disorder is NK-LGLL and the antibody specifically binds to human NKG2A.

Also provided herein is a method for treating CLPD-NK in a human subject in need thereof, comprising administering to the human subject an effective amount of an antibody, wherein the antibody specifically binds to human NKG2A, and wherein the antibody comprises a human immunoglobulin Fc region comprising enhanced ADCC activity compared to a wild type IgG1 Fc region. In some embodiments, administration of the antibody to the human subject results in an improvement of CLPD-NK symptoms in the human.

Also provided herein is a method for treating CLPD-NK in a human subject in need thereof, comprising administering to the human subject an effective amount of an antibody, wherein the antibody specifically binds to human CD94, and wherein the antibody comprises a human immunoglobulin Fc region comprising enhanced ADCC activity compared to a wild type IgG1 Fc region. In some embodiments, administration of the antibody to the human subject results in an improvement of CLPD-NK symptoms in the human.

In some embodiments, the terms treat, treating, treatment, ameliorate, ameliorating, reducing one or more symptoms, reducing symptoms, reduce one or more symptoms, reduce symptoms, and other grammatical equivalents, refer to alleviating, abating or ameliorating one or more symptoms of a disease or disorder, preventing additional symptoms, ameliorating or preventing the underlying causes of symptoms, inhibiting the disease or disorder, e.g., arresting the development of the disease or disorder, relieving the disease or disorder, causing regression of the disease or disorder, relieving a condition caused by the disease or disorder, or stopping the symptoms of the disease or disorder, and are intended to include prophylaxis. In some embodiments, the terms further include achieving a therapeutic benefit and/or a prophylactic benefit. In some embodiments, a therapeutic benefit refers to eradication or amelioration of the underlying disease or disorder being treated. Also, a therapeutic benefit is achieved with the eradication or amelioration of one or more of the physiological symptoms associated with the underlying disease or disorder such that an improvement is observed in the patient, notwithstanding that, in some embodiments, the patient is still afflicted with the underlying disease or disorder. For prophylactic benefit, the pharmaceutical compositions are administered to a patient at risk of developing a particular disease or disorder, or to a patient reporting one or more of the physiological symptoms of a disease or disorder, even if a diagnosis of the disease or disorder has not been made.

In some embodiments, an effective amount, a therapeutically effective amount or pharmaceutically effective amount may be a sufficient amount of at least one pharmaceutical composition or compound (e.g., an antibody of the disclosure) being administered which will relieve to some extent one or more of the symptoms of the disease or condition being treated.

In some embodiments, at least about 2,000 receptors per cell (e.g., any of at least about 1,000 receptors per cell, at least about 2,000 receptors per cell, at least about 3,000 receptors per cell, at least about 4,000 receptors per cell, at least about 5,000 receptors per cell, or at least about 7,000 receptors per cell, at least about 10,000 receptors per cell, at least about 20,000 receptors per cell, at least about 30,000 receptors per cell, at least about 40,000 receptors per cell, at least about 50,000 receptors per cell, at least about 60,000 receptors per cell, at least about 70,000 receptors per cell, at least about 80,000 receptors per cell, at least about 90,000 receptors per cell, at least about 100,000 receptors per cell, at least about 200,000 receptors per cell, at least about 300,000 receptors per cell, at least about 400,000 receptors per cell, at least about 500,000 receptors per cell, at least about 600,000 receptors per cell, at least about 700,000 receptors per cell, at least about 800,000 receptors per cell, at least about 900,000 receptors per cell, at least about 1,000,000 receptors per cell, or more) of the cell surface protein (e.g., human CD94, human CD57, or human NKG2A) are expressed on the surface of the peripheral blood LGL and/or NK cells in the subject. In some embodiments, the number of receptors of a cell surface protein (e.g., human CD94, human CD57, or human NKG2A) on the surface of the peripheral blood LGL and/or NK cells is compared between a sample (e.g., a biospecimen) obtained from a healthy (e.g., normal) subject and a sample obtained from a subject with a disease or disorder (e.g., NK cells (CLPD-NK), LGL leukemia, Felty's syndrome, rheumatoid arthritis, aggressive NK leukemia, inclusion body myositis, or inflammatory bowel disease). In some embodiments, expression of a cell surface protein (e.g., human CD94, human CD57, or human NKG2A) is specific to the surface of LGL and/or NK cells. In some embodiments, expression of a cell surface protein (e.g., human CD94, human CD57, or human NKG2A) is specific to the surface of LGL and/or NK cells in a sample from a subject with a disease or disorder (e.g., human CD94, human CD57, or human NKG2A). The number of cell surface proteins (e.g., receptors) expressed on the surface of the peripheral blood LGL and/or NK cells in the subject may be measured using any method known in the art, such as flow cytometry, e.g., as described in Examples 1-3. In some embodiments, by using the same biospecimens we will show expression of CD94 or CD57 or NKG2A and additional cell surface protein that is specific for LGL cells.

In some embodiments, the reduction in the number of peripheral blood LGL and/or NK cells in the subject occurs within the first 24 hours, e.g., any of within about 1 hour, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, or within about 24 hours after administration of the antibody to the subject.

In some embodiments, the number of peripheral blood LGL and/or NK cells in the subject (e.g., in a peripheral blood sample obtained from the subject) is reduced to below the limit for clinical diagnosis of the disease or disorder. In some embodiments, the number of peripheral blood LGL and/or NK cells in the subject is reduced to less than or equal to 2×10⁹ cells/L (e.g., in a peripheral blood sample obtained from the subject). See, e.g., Lamy, T. et al. (2017) Blood 129:1082-1094. In some embodiments, the reduction in the number of peripheral blood LGL and/or NK cells in the subject to below the limit for clinical diagnosis of the disease or disorder is present in the subject for at least about 1 week, e.g., any of at least about 1 week, at least about 2 weeks, at least about 3 weeks, at least about 4 weeks, at least about 1 month, at least about 2 months, at least about 3 months, at least about 4 months, at least about 5 months, at least about 6 months, or more, after administration of the antibody to the subject. In some embodiments, the reduction in the number of peripheral blood LGL and/or NK cells in the subject to less than or equal to 2×10⁹ cells/L in the subject (e.g., in a peripheral blood sample obtained from the subject) for at least about 1 week, e.g., any of at least about 1 week, at least about 2 weeks, at least about 3 weeks, at least about 4 weeks, at least about 1 month, at least about 2 months, at least about 3 months, at least about 4 months, at least about 5 months, at least about 6 months, or more, after administration of the antibody to the subject.

In some embodiments, the number of peripheral blood LGL and/or NK cells in the subject is reduced to below the limit of detection for the peripheral blood LGL and/or NK cells in the subject. In some embodiments, the reduction in the number of peripheral blood LGL and/or NK cells in the subject to below the limit of detection for the peripheral blood LGL and/or NK cells is present in the subject for at least about 1 week, e.g., any of at least about 1 week, at least about 2 weeks, at least about 3 weeks, at least about 4 weeks, at least about 1 month, at least about 2 months, at least about 3 months, at least about 4 months, at least about 5 months, at least about 6 months, or more, after administration of the antibody to the subject. In some embodiments, peripheral blood LGL and/or NK cells are detected by flow cytometry (e.g., as performed on a peripheral blood sample from the subject) using the following markers: CD3-CD8-CD16+CD56+CD57+ (for CLPD-NK immunophenotype) or CD3+CD8+CD16+CD56-CD57+ (for T-LGLL immunophenotype).

In some embodiments, the reduction in the number of peripheral blood LGL and/or NK cells in the subject is reversible. In some embodiments, the reduction in the number of peripheral blood LGL and/or NK cells in the subject is reversible within any of about 1 week, about 2 weeks, about 3 weeks, about 4 weeks, about 1 month, about 2 months, about 3 months, about 4 months, about 5 months, about 6 months, or more, after administration of the antibody to the subject.

In some embodiments, administration of the antibody to the subject results in a reduction in the number of peripheral blood LGL and/or NK cells in the subject. In some embodiments, the NK cells in the subject are CD3 negative and CD56 positive, CD3 negative and CD16 positive, CD3 negative and CD57 positive, CD3 negative and CD94 positive, or CD3 negative and NKG2A positive. In some embodiments, the NK cells in the subject are CD3 negative and CD56 positive. In some embodiments, the NK cells in the subject are CD3 negative and CD16 positive. The biomarkers expressed by the NK cells (e.g., CD3, CD16, CD56) may be measured using any method known in the art, such as flow cytometry, e.g., as described in Examples 1-3.

In some embodiments, a statement that a cell or a population of cells is positive (+) for, or expresses a particular marker (e.g., CD3, CD4, CD8, CD16, CD56, CD57, CD94, NKG2A, etc.), refers to the detectable presence on or in the cell of the particular marker. In some embodiments, a statement that a cell or a population of cells is positive for, +, or expresses a surface marker (e.g., a cell surface protein) refers to the presence of cell surface expression of the particular marker, for example, as detected by flow cytometry, for example, by staining with an antibody that specifically binds to the marker and detecting said antibody, wherein the staining is detectable by flow cytometry at a level substantially above the staining detected carrying out the same procedure with an isotype-matched control and/or fluorescence minus one (FMO) gating control under otherwise identical conditions, and/or at a level substantially similar to that for cell known to be positive for the marker, and/or at a level substantially higher than that for a cell known to be negative for the marker.

In some embodiments, a statement that a cell or a population of cells is negative (−) for, or does not express a particular marker (e.g., CD3, CD4, CD8, CD16, CD56, CD57, CD94, NKG2A, etc.) refers to the absence of a detectable presence on or in the cell of the particular marker. In some embodiments, a statement that a cell or a population of cells is negative for, −, or does not express a surface marker (e.g., a cell surface protein) refers to the absence of cell surface expression of the particular marker, for example, as detected by flow cytometry, for example, by staining with an antibody that specifically binds to the marker and detecting said antibody, wherein the staining is detectable by flow cytometry at a level substantially similar or below the staining detected carrying out the same procedure with an isotype-matched control and/or fluorescence minus one (FMO) gating control under otherwise identical conditions, and/or at a level below that for cell known to be positive for the marker, and/or at a level substantially similar or below that for a cell known to be negative for the marker.

In some embodiments, the antibody has an EC50 for reducing peripheral blood LGL and/or NK cells in the subject of between about 1 ng/ml and about 100 ng/ml, e.g., any of about 1 ng/ml, about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, or about 100 ng/ml. In some embodiments, the antibody has an EC50 of between about 3 ng/ml and about 40 ng/ml. In some embodiments, the antibody has an EC50 of about 3 ng/ml. In some embodiments, the antibody has an EC50 of about 40 ng/ml. EC50 may be measured using any method known in the art, e.g., as described in the Examples.

In some embodiments, administration of the antibody to the subject does not result in a reduction of T cells in the subject. In some embodiments, the T cells in the subject are CD3 positive and CD4 positive or CD3 positive and CD16 negative. The biomarkers expressed by the T cells (e.g., CD3, CD16, CD4) may be measured using any method known in the art, such as flow cytometry, e.g., as described in the Examples.

In some embodiments, the subject is a human, a primate, a non-human primate (e.g., African green monkeys, rhesus monkeys, etc.), a farm mammal, a game mammal, or a domestic mammal. In some embodiments, the subject is a human. In some embodiments, the human subject is an infant, a toddler, a child, a young adult, an adult or a geriatric. In some embodiments, the subject has a disease involving LGLs and/or NK cells, e.g., CLPD-NK, LGL leukemia, Felty's syndrome, rheumatoid arthritis, aggressive NK leukemia, inclusion body myositis, or inflammatory bowel disease.

In some embodiments, administration of the antibody to the subject does not result in tumor lysis syndrome in the subject. Tumor lysis syndrome may be measured or diagnosed according to any method known in the art, such as the Cairo-Bishop classification system for tumor lysis syndrome (see, e.g., Cairo and Bishop (2004) Br J Haematol, 127(1):3-11.)

In some embodiments, an antibody of the disclosure binds to CD94 or CD57 or NKG2A. In some embodiments, an antibody of the disclosure depletes and/or reduces the level of LGL and/or NK cells. In some embodiments, an antibody of the disclosure has clear benefits for a patient (e.g., a human patient) having a disease or disorder, such as CLPD-NK, LGL leukemia, rheumatoid arthritis, Felty's syndrome, aggressive NK leukemia, IBM, IBD, and other diseases associated with LGL and/or NK cells. In some embodiments, an antibody of the disclosure has better tolerability and fewer side effects over the first and second line of therapies for the disease or disorder (e.g., CLPD-NK, LGL leukemia, Felty's syndrome, rheumatoid arthritis, aggressive NK leukemia, inclusion body myositis, or inflammatory bowel disease), such as chemotherapy, Alemtuzumab, and splenectomy. In some embodiments, an antibody of the disclosure demonstrates more selective depletion of the disease-inducing cells (e.g., peripheral blood LGL and/or NK cells) compared to current therapies that are non-selective, such as chemotherapy, Alemtuzumab, and splenectomy. Accordingly, in some embodiments, the disclosure provides a method of reducing the number or depleting LGL and/or NK cells in a human subject upon administration of molecule (e.g., an antibody of the disclosure) that binds to cell surface protein on LGL and/or NK cells, such as CD94 or CD57or NKG2A, or an additional cell surface protein that is specific for LGL and/or NK cells, and that comprises (a) a region that specifically binds to the target and (b) an immunoglobulin Fc region.

A. Administration and Dosing Regimens

(i) Routes of Administration

In some embodiments, administer, administering, administration, and the like, refer to methods that are used to enable delivery of therapeutic or pharmaceutical compositions to the desired site of biological action. In some embodiments, an antibody of the disclosure (and any additional therapeutic agent) for use in any of the methods provided herein may be administered to the subject (e.g., a human) by any suitable means, including parenteral, intrapulmonary, intranasal, and intralesional administration. Parenteral infusions include intramuscular, intravenous, intraarterial, intraperitoneal, or subcutaneous administration. In some embodiments, an antibody of the disclosure is administered by intravenous infusion. Dosing of an antibody of the disclosure can be by any suitable route, e.g., by injections, such as intravenous or subcutaneous injections, depending in part on whether the administration is brief or chronic.

(ii) Dosing Regimens

An antibody of the disclosure for use in any of the methods provided herein may be administered to the subject using various dosing schedules or regimens, including but not limited to single or multiple administrations over various time-points, bolus administration, and pulse infusion. The specific dosage of the antibodies of the disclosure to be administered will vary according to the particular target specificity, the type of disease or disorder, the subject, and the nature and severity of the disease, the physical condition of the subject, the therapeutic regimen (e.g., whether a combination therapeutic agent is used), and the selected route of administration. In some embodiments, a dose of an antibody of the disclosure may range from about 0.0001 mg/kg to 100 mg/kg of the subject's body weight. An exemplary dosage regimen of an antibody of the disclosure entails administration of the antibody in multiple dosages over a prolonged period, for example, of at least six months.

B. Diseases

There are 3 distinct diseases involving LGLs: T-cell LGL (T-LGL) leukemia; chronic lymphoproliferative disorders of NK cells (CLPD-NK, formerly NK-LGL); and aggressive NK-cell leukemias, such as aggressive natural killer leukemia (ANKL) and extranodal NKL nasal type (ENKL).

In addition to the NK or T LGL leukemias, NK or LGL cells play key roles in rheumatoid arthritis (RA), Felty's syndrome, aggressive NK leukemia, Inclusion body myositis (IBM), inflammatory bowel disease (IBD), and other diseases. Non-limiting examples of diseases and disorders in which LGL and NK cells play a role include LGL leukemia, Rheumatoid arthritis, Felty's syndrome, aggressive NK leukemia, IBM, and IBD. Advantageously, the methods described herein may be used, e.g., to reduce the number of abnormal or pathologic NK cells (e.g., CLPD-NK, ANKL, or ENKL cells) via mechanisms such as ADCC that employ NK cells, essentially using the pathologic cells to eliminate each other. For exemplary descriptions of symptoms of these diseases, see, e.g., Lamy, et al, Blood, 2017 x Vol. 129, No. 9; Loughran Blood, VOI 82, NO 1 (July I), 1993: pp 1-14; Semenzato G, et al, Blood. 1997; 89(1):256-260; and Bourgault-Rouxel, et al, Leuk Res. 2008; 32(1):45-48.

(i) CLPD-NK

Chronic lymphoproliferative disorders of NK cells (CLPD-NK), also referred to as NK-LGL leukemia, chronic NK cell lymphocytosis, chronic NK-LGL lymphoproliferative disorder (LPD), NK cell lineage granular lymphocyte proliferative disorder, NK cell LGL lymphocytosis, or indolent granular NK cell LPD is generally characterized by a persistent (e.g., 6 months or greater) increase in peripheral blood NK cells (e.g., ≥2×10⁹/L).

Symptoms of CLPD-NK include variable cytopenias such as neutropenia and anemia, fatigue, fever, night sweats, recurrent infections, rheumatoid arthritis, lymphadenopathy, hepatosplenomegaly, skin lesions, hematologic neoplasms, vasculitis, neuropathy, and autoimmune disorders.

In some embodiments of the methods provided herein, the disease or disorder is CLPD-NK, and administration of the antibody results in a reduction in one or more CLPD-NK symptoms in the subject. In some embodiments, the reduction in the number of peripheral blood LGL and/or NK cells in the subject after administration of the antibody results in a reduction in one or more CLPD-NK symptoms in the subject.

Symptoms of CLPD-NK may be measured by any method known in the art, such as using laboratory tests to measure anemia, neutropenia, complete blood counts, and/or magnetic resonance imaging (MRI), CT scan, palpation, or ultrasound (e.g., to determine hepatosplenomegaly), bone marrow exams, and flow cytometry. Methods for measuring symptoms of CLPD-NK are described, e.g., in Swerdlow, S. H. et al. (2016) Blood 127:2375-2390.

(ii) LGL Leukemia

Large granular lymphocytic (LGL) leukemia is a chronic lymphoproliferative disorder that exhibits a chronic elevation in large granular lymphocytes (LGLs) in the peripheral blood and is called T-cell LGL leukemia.

Symptoms of LGL leukemia include splenomegaly, B symptoms (e.g., systemic symptoms such as fever, night sweats, and weight loss), anemia, neutropenia, and recurrent infections. Rheumatoid arthritis is often also found in people with T-cell LGL leukemia.

In some embodiments of the methods provided herein, the disease or disorder is LGL leukemia, and administration of the antibody results in a reduction in one or more LGL leukemia symptoms in the subject. In some embodiments, the reduction in the number of peripheral blood LGL and/or NK cells in the subject after administration of the antibody results in a reduction in one or more LGL leukemia symptoms in the subject.

Symptoms of LGL leukemia may be measured by any method known in the art, such as using laboratory tests to measure anemia, neutropenia, and other cytopenias, complete blood counts, magnetic resonance imaging (MRI), CT scan, palpation, or ultrasound (e.g., to determine splenomegaly), bone marrow exams, and flow cytometry. Methods for measuring symptoms of LGL leukemia are described, e.g., in Swerdlow, S. H. et al. (2016) Blood 127:2375-2390.

(iii) Felty's Syndrome

Felty's syndrome is an autoimmune disease characterized by rheumatoid arthritis, splenomegaly (e.g., inflammatory splenomegaly), and a reduced number of neutrophils in the blood. Symptoms of Felty's syndrome include painful, stiff, and/or swollen joints, physical findings associated with rheumatoid arthritis, splenomegaly, neutropenia, infections, keratoconjunctivitis sicca, fever, weight loss, fatigue, discoloration of the skin, sores (e.g., ulcers), hepatomegaly, anemia, thrombocytopenia, abnormal liver function, enlarged lymph nodes, and vasculitis.

In some embodiments of the methods provided herein, the disease or disorder is Felty's syndrome, and administration of the antibody results in a reduction in one or more Felty's syndrome symptoms in the subject. In some embodiments, the reduction in the number of peripheral blood LGL and/or NK cells in the subject after administration of the antibody results in a reduction in one or more Felty's syndrome symptoms in the subject. Symptoms of Felty's syndrome include, without limitation, joint inflammation, joint pain, and splenomegaly.

Symptoms of Felty's syndrome may be measured by any method known in the art, such as using laboratory tests to measure anemia, neutropenia, thrombocytopenia, and other cytopenias, complete blood counts, magnetic resonance imaging (MRI), CT scan, or ultrasound (e.g., to determine splenomegaly and/or hepatomegaly), laboratory tests for abnormal liver function, palpation to determine splenomegaly and/or hepatomegaly, flow cytometry, disease activity score-28 (DAS-28, e.g., as used for monitoring rheumatoid arthritis symptoms), and DAS-28 with erythrocyte sedimentation rate (ESR).

(iv) Rheumatoid Arthritis

Rheumatoid arthritis is an autoimmune disorder that primarily affects the joints, but can also affect other organs and can be associated with cardiovascular disease, osteoporosis, interstitial lung disease, infection, cancer, fatigue, and depression. Symptoms of rheumatoid arthritis include swollen, tender, and warm joints, joint inflammation, joint pain, joint stiffness, splenomegaly, rheumatoid nodules (e.g., in the skin), necrotizing granuloma, vasculitis, pyoderma gangrenosum, Sweet's syndrome, drug reactions, erythema nodsum, lobe pannicultis, atrophy of finger skin, palmar erythema, skin fragility, diffuse alopecia areata, lung fibrosis, Caplan's syndrome, exudative pleural effusions, atherosclerosis, myocardial infarction, stroke, pericarditis, endocarditis, left ventricular failure, valvulitis, fibrosis of the heart and/or blood vessels, anemia, increased platelet count, low white blood cell count, renal amyloidosis, episcleritis, scleritis, keratoconjuctivitis sicca, keratitis, loss of vision, liver problems, peripheral neuropathy, mononeuritis multiplex, carpal tunnel syndrome, myelopathy, atlanto-axial subluxation, vertebrae slipping, fatigue, low grade fever, malaise, morning stiffness, loss of appetite, loss of weight, osteoporosis, cancer (e.g., lymphoma, skin cancer), and periodontitis.

In some embodiments of the methods provided herein, the disease or disorder is rheumatoid arthritis, and administration of the antibody results in a reduction in one or more rheumatoid arthritis symptoms in the subject. In some embodiments, the reduction in the number of peripheral blood LGL and/or NK cells in the subject after administration of the antibody results in a reduction in one or more rheumatoid arthritis symptoms in the subject.

In some embodiments, symptoms and disease status/progression of rheumatoid arthritis are measured according to the 2010 ACR/EULAR Rheumatoid Arthritis Classification Criteria (see, e.g., Aletaha et al., (2010) Annals of Rheumatic Diseases, 69(9):1580-8). Symptoms of rheumatoid arthritis may also be measured by any method known in the art, such as using laboratory tests to measure erythrocyte sedimentation rates, C-reactive protein, rheumatoid factor, anti-citrullinated protein antibodies, anemia and other cytopenias, increased platelet count, low white blood cell count, complete blood counts, renal amyloidosis, medical imaging such as X-rays, MRI, CT-scans, ultrasound (e.g., ultrasonography using a high-frequency transducer; Doppler ultrasound), flow cytometry, disease activity score-28 (DAS-28), and DAS-28 with erythrocyte sedimentation rate (ESR).

(v) Aggressive NK Leukemia

Aggressive NK-cell leukemia is an aggressive disease with systemic proliferation of NK cells and a rapidly declining clinical course. Aggressive NK leukemia may also be referred to as aggressive NK-cell lymphoma. Symptoms of aggressive NK-cell leukemia include constitutional symptoms (e.g., malaise, weight loss, fatigue), hepatosplenomegaly, lymphadenopathy, coagulopathies, hemophagocytic syndrome, multi-organ failure, infections such as Epstein-Barr virus, allergic reactions (e.g., allergic reactions to insect bites, such as mosquito bites) that may result in necrosis and systemic symptoms such as fever, swollen lymph nodes, abdominal pain, diarrhea, and anaphylaxis.

In some embodiments of the methods provided herein, the disease or disorder is aggressive NK-cell leukemia, and administration of the antibody results in a reduction in one or more aggressive NK-cell leukemia symptoms in the subject. In some embodiments, the reduction in the number of peripheral blood LGL and/or NK cells in the subject after administration of the antibody results in a reduction in one or more aggressive NK-cell leukemia symptoms in the subject.

Symptoms of aggressive NK leukemia may be measured by any method known in the art, such as using laboratory tests, e.g., to measure anemia, neutropenia, and other cytopenias, complete blood counts, and/or magnetic resonance imaging (MRI), CT scan, palpation, or ultrasound (e.g., to determine splenomegaly), bone marrow exams, and flow cytometry. Methods for measuring symptoms of aggressive NK leukemia are described, e.g., in Swerdlow, S. H. et al. (2016) Blood 127:2375-2390.

(vi) Inclusion Body Myositis

Inclusion Body Myositis (IBM), also referred to as sporadic inclusion body myositis, is an inflammatory muscle disease characterized by autoimmune and degenerative processes that result in progressive weakness and wasting of distal and/or proximal muscles. Generally, IBM is characterized by invasion of immune cells into muscle tissues. In some cases, patients with IBM have elevated creatine kinase levels in the blood. Symptoms of IBM include progressive muscle weakness, muscle wasting/atrophy, frequent tripping and falling, difficulty manipulating fingers, foot drop, restricted mobility, impaired balance, muscle pain, dysphagia, and fatigue.

In some embodiments of the methods provided herein, the disease or disorder is IBM, and administration of the antibody results in a reduction in one or more IBM symptoms in the subject. In some embodiments, the reduction in the number of peripheral blood LGL and/or NK cells in the subject after administration of the antibody results in a reduction in one or more IBM symptoms in the subject.

Symptoms of IBM may be measured by any method known in the art, such as muscle biopsies, blood tests (e.g., to measure creatine kinase), electromyography (EMG) studies, blood tests to measure antibodies to NT5C1A, flow cytometry, and myositis disease activity assessment tools including without limitation Myositis Intention to Treat Activity Index (MITAX) and Myositis Disease Activity Assessment Visual Analogue Scales (MYOACT).

(vii) Inflammatory Bowel Disease

Inflammatory bowel disease (IBD) refers to a class of inflammatory conditions of the colon and small intestine. Types of IBD include ulcerative colitis and Crohn's disease. Symptoms of IBD include diarrhea, fever, fatigue, abdominal pain, abdominal cramping, blood in the stool, reduced appetite, and weight loss.

In some embodiments of the methods provided herein, the disease or disorder is IBD, and administration of the antibody results in a reduction in one or more IBD symptoms in the subject. In some embodiments, the reduction in the number of peripheral blood LGL and/or NK cells in the subject after administration of the antibody results in a reduction in one or more IBD symptoms in the subject.

Symptoms of IBD may be measured by any method known in the art, such as laboratory blood tests for anemia, other cytopenias, or infections, fecal occult blood tests, colonoscopies, flexible sigmoidoscopy, upper endoscopy, capsule endoscopy, balloon-assisted enteroscopy, X-rays, CT-scans, MRI scans, ultrasound, and flow cytometry.

### II. Antibodies

In some embodiments, provided herein are molecules (e.g., antibodies) that bind to CD94, CD57, NKG2A, or other cell surface proteins expressed on LGL and/or NK cells. Also provided herein are molecules (e.g., antibodies) that bind to CD94 or CD57 or NKG2A and that have immunoglobulin Fc part with modifications including reduced fucosylation, non-fucosylation or mutations that enhance ADCC activities and/or improve affinity of the Fc region to Fc receptors such as CD16.

In some embodiments, the antibodies provided herein bind to human CD94, human CD57, or human NKG2A. In some embodiments, the antibodies provided herein bind to CD94, CD57, or NKG2A.

In some embodiments, the term antibody is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. In some embodiments, an antibody of the disclosure is an isolated antibody. An “isolated” antibody is one which has been identified and separated and/or recovered from a component of its natural environment. Contaminant components of its natural environment are materials which would interfere with research, diagnostic, and/or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

In some embodiments, a monoclonal antibody is an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, in some embodiments, a monoclonal antibody is obtained from a substantially homogeneous population of antibodies. Monoclonal antibodies may be produced using any method known in the art. For example, monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci.

A. Enhanced ADCC Activity

In some embodiments, antibody-dependent cell-mediated cytotoxicity, antibody-dependent cellular cytotoxicity, antibody directed cell cytotoxicity, or ADCC refer to a cell-mediated reaction in which non-specific cytotoxic cells producing Fc receptors, e.g. natural killer cells (NK cells), neutrophils, and macrophages, recognize an antibody bound to a target cell and then cause lysis of the target cell. The primary mediator cells are natural killer (NK) cells. NK cells express FcγRIII (Ravetch et al. (1991) Annu. Rev. Immunol., 9:457-92). In some embodiments, ADCC activity refers to the ability of an antibody or Fc fusion protein to elicit an ADCC reaction.

In some embodiments, the antibodies provided herein have enhanced antibody-dependent cellular cytotoxicity (ADCC) activity. In some embodiments, enhanced ADCC activity refers to an antibody or an Fc region of an antibody mediating or inducing ADCC more efficiently and/or more effectively than a native or wild type antibody and/or a native or wild type Fc region of an antibody in the presence of effector cells in vitro or in vivo, which may be determined using an ADCC assay, e.g., as described herein or as is commonly known in the art. In some embodiments, effector cells are leukocytes that produce one or more Fc receptors and perform effector functions. In some embodiments, such cells produce at least FcγRIII and perform the ADCC effector function. Examples of ADCC-mediated human leukocytes include peripheral blood mononuclear cells (PBMCs), natural killer cells (NK), monocytes, cytotoxic T cells, and neutrophils.

In some embodiments, ADCC activity can be assessed directly using an in vitro assay, e.g., as described in the Examples, using a ⁵¹Cr release assay using peripheral blood mononuclear cells (PBMC) and/or NK effector cells, see e.g., Shields et al. (2001) J. Biol. Chem., 276:6591-6604, or another suitable method. ADCC activity may be expressed as the number of remaining cells following an ADCC assay (see, e.g., Example 2), or a concentration of antibody or Fc fusion protein at which the lysis of target cells is half-maximal (e.g., EC50). In some embodiments, ADCC activity is determined using an ex vivo assay using PBMCs and/or NK cells, e.g., as described in the Examples, and the ADCC activity of an antibody of the disclosure is described as the percent of target cells remaining after the ADCC assay and/or the EC50 of the antibody (i.e., the concentration of an antibody of the disclosure at which half the maximum target cell depletion or cell lysis is achieved). The EC50 of antibody may be determined using any method known in the art, e.g., using a dosage response curve and GraphPad Prism, e.g., as described in the Examples. In some embodiments, the antibodies provided herein induce ADCC activity with an EC50 measured using an ex vivo assay, e.g., as described in the Examples, of between about 1 ng/ml to about 100 ng/ml (e.g., any of about 1 ng/ml, about 2 ng/ml, about 3 ng/ml, about 4 ng/ml, about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, or about 100 ng/ml). In some embodiments, an antibody of the disclosure exhibits an EC50 that is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than the EC50 of a control antibody (e.g., a wild type control antibody, or an antibody known in the art or commercially available against the same target).

In some embodiments, EC50 refers to the concentration of a compound (e.g., an antibody) which induces a response halfway between the baseline and maximum after a specified exposure time. For example, EC50 may be used to measure the potency of an antibody for mediating and/or inducing an effector function, e.g., ADCC activity. In some embodiments, the EC50 of a dose response curve represents the concentration of a compound (e.g., an antibody) where 50% of its maximal effect is observed.

In some embodiments, an antibody of the disclosure has a higher maximal target cell lysis compared to a control antibody (e.g., a wild type control antibody, or an antibody known in the art or commercially available against the same target). For example, antibodies of the disclosure may exhibit a maximal target cell lysis that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% higher than that of a control antibody (e.g., a wild type control antibody, or an antibody known in the art or commercially available against the same target).

(i) Enhanced Binding to Fc Receptors

In some embodiments, the antibodies provided herein include a human immunoglobulin Fc region that has enhanced ADCC activity compared to a wild type Fc region. In some embodiments, the antibodies provided herein bind to a human cellular Fc receptor to a greater extent than an antibody comprising a wild type Fc region. In some embodiments, an Fc receptor (FcR) is a receptor that is capable of binding to an Fc region of an antibody. Certain Fc receptors can bind to IgG (i.e., γ-receptor); such receptors include subclasses of FcyRI, FcyRII and FcyRIII, as well as their allelic variants and alternative splicing events. For an overview of the Fc receptors see Ravetch and China: Annu. Port. Immunol. 9, 457 (1991); Capel et al. Immunomethods, 4, 25 (1994); and de Haas et al., J. Leg. Clin. Med. 126, 330 (1995).

In some embodiments, the antibodies provided herein bind to a human cellular Fc gamma receptor IIIA to a greater extent than an antibody comprising a wild type Fc region. In some embodiments, the human cellular Fc gamma receptor IIIA comprises a valine residue or a phenylalanine residue at amino acid residue position 158. See, e.g., UniProt Accession P08637 or VAR_003960. In some embodiments, the human cellular Fc gamma receptor IIIA comprises the sequence of SEQ ID NO: 8 or 9.

Human cellular Fc gamma receptor IIIA 158F
(SEQ ID NO: 8)
MWQLLLPTALLLLVSAGMRTEDLPKAVVFLEPQWY
RVLEKDSVTLKCQGAYSPEDNSTQWFHNESLISSQ
ASSYFIDAATVDDSGEYRCQTNLSTLSDPVQLEVH
IGWLLLQAPRWVFKEEDPIHLRCHSWKNTALHKVT
YLQNGKGRKYFHHNSDFYIPKATLKDSGSYFCRGL
FGSKNVSSETVNITITQGLAVSTISSFFPPGYQVS
FCLVMVLLFAVDTGLYFSVKTNIRSSTRDWKDHKF
KWRKDPQDK
Human cellular Fc gamma receptor IIIA 158V
(SEQ ID NO: 9)
MWQLLLPTALLLLVSAGMRTEDLPKAVVFLEPQWY
RVLEKDSVTLKCQGAYSPEDNSTQWFHNESLISSQ
ASSYFIDAATVDDSGEYRCQTNLSTLSDPVQLEVH
IGWLLLQAPRWVFKEEDPIHLRCHSWKNTALHKVT
YLQNGKGRKYFHHNSDFYIPKATLKDSGSYFCRGL
VGSKNVSSETVNITITQGLAVSTISSFFPPGYQVS
FCLVMVLLFAVDTGLYFSVKTNIRSSTRDWKDHKF
KWRKDPQDK

In some embodiments, an antibody provided herein is of the IgG (e.g., IgG1, IgG2, IgG3, or IgG4), IgA (IgA1 or IgA2), IgD, IgM, or IgE isotype. In some embodiments, an antibody provided herein is of the IgG, isotype. In some embodiments, an antibody provided herein is of the IgG1, isotype. In some embodiments, antibodies provided herein bind to a human cellular Fc gamma receptor IIIA (FcγRIIIA) to a greater extent than an antibody comprising a wild type human IgG1 Fc region. In some embodiments, the human cellular Fc gamma receptor IIIA comprises a valine residue or a phenylalanine residue at amino acid residue position 158. Exemplary assays for determining binding to a human cellular Fc gamma receptor IIIA are known in the art; see, e.g., Lazar, G. A. et al. (2006) Proc. Natl. Acad. Sci. 103:4005-1010; and Ferrara, C. et al. (2011) Proc. Natl. Acad. Sci. 108:12669-12674.

In some embodiments, an Fc region is a C-terminal region of an immunoglobulin heavy chain that contains at least a portion of the constant region. In some embodiments, an Fc region includes a native Fc region or a variant Fc region. In one embodiment, a human IgG heavy chain Fc region extends from Cys226, or from Pro230, to the carboxyl-terminus of the heavy chain. However, the C-terminal lysine (Lys447) of the Fc region may or may not be present. In some emobdiments, numbering of amino acid residues in an Fc region or constant region is according to the EU numbering system, also called the EU index, as described in Kabat et al., Sequences of Proteins of Immunological Interest, 5th Ed. Public Health Service, National Institutes of Health, Bethesda, Md., 1991. In some embodiments, a wild type Fc region or a native Fc region are an Fc region that comprises an amino acid sequence that is identical to the amino acid sequence of the Fc region found in nature. In some embodiments, a variant Fc region is an Fc region that comprises an amino acid sequence that differs from the native or wild type sequence of the Fc region in at least one amino acid. In some embodiments, a variant Fc region has at least one amino acid substitution, e.g., approximately 1-10 or 1-5 amino acid substitutions. In some embodiments, the Fc region variant is at least approximately 80% (e.g., at least about 90%, or at least about 95%) homologous to a native or wild type sequence Fc region and/or an Fc region of an original polypeptide. In some embodiments, the at least one amino acid substitution in the variant Fc region enhances the effector function of the variant Fc region compared to a native or wild type Fc region. In some embodiments, an effector function is a biological activity attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); antibody-dependent cell-mediated phagocytosis (ADCP); down regulation of cell surface receptors (e.g., B-cell receptor); and B-cell activation.

The binding affinity of an antibody for an Fc receptor may be assessed using any method known in the art, such as using surface plasmon resonance, and/or ELISA, e.g., as described in Shields et al. (2001) J. Biol. Chem., 276:6591-6604. In some embodiments, the affinity of an antibody of the disclosure for FcγRIIIA may be above that of the wild-type control by any of at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20 fold, at least about 30-fold, at least about 40-fold, at least about 50-fold, or higher.

In some embodiments, affinity refers to the strength of the sum total of noncovalent interactions between a single binding site of a molecule (e.g., an antibody) and its binding partner (e.g., an antigen or a target). For example, the affinity of a molecule X for its partner Y can generally be represented by the dissociation constant (K_{D}). Affinity can be measured by common methods known in the art, including those described herein.

In some embodiments, statements that a molecule (e.g., an antibody and/or an F_{c} region) binds to a greater extent than another molecule (e.g., an antibody and/or an Fc region), or that a molecule (e.g., an antibody and/or an Fc region) binds with a greater affinity than another molecule (e.g., an antibody and/or an Fc region), or other grammatical equivalents, refer to a molecule (e.g., an antibody and/or an Fc region) binding more tightly (e.g., having a lower dissociation constant) to a target (e.g., an Fc receptor, a cell surface protein) than another molecule (e.g., an antibody and/or an Fc region) in binding assays (e.g., as described herein and/or as commonly known in the art) under substantially the same conditions. For example, the statement that an antibody “X” binds to an Fc receptor to a greater extent than an antibody “Y” indicates that antibody “X” binds more tightly (e.g., has a lower dissociation constant) to an Fc receptor than antibody “Y” in binding assays (e.g., as described herein and/or as commonly known in the art) under substantially the same conditions. In another example, the statement that an antibody “X” binds to a target (e.g., a cell surface protein) with a greater affinity than an antibody “Y” indicates that antibody “X” binds more tightly (e.g., has a lower dissociation constant) to a target (e.g., a cell surface protein) than antibody “Y” in binding assays (e.g., as described herein and/or as commonly known in the art) under substantially the same conditions.

(ii) Reduced Fucosylation

In some embodiments, an antibody of the present disclosure is non-fucosylated or fucose-deficient, e.g., a glycosylation antibody variant comprising an Fc region wherein a carbohydrate structure attached to the Fc region has reduced fucose or lacks fucose. In some embodiments, an antibody with reduced fucose or lacking fucose has improved ADCC function. Non-fucosylated or fucose-deficient antibodies have reduced fucose relative to the amount of fucose on the same antibody produced in a cell line. In some embodiments, a non-fucosylated or fucose-deficient antibody composition of the present disclosure is a composition in which less than about 50% of the N-linked glycans attached to the Fc region of the antibodies in the composition comprise fucose.

In some embodiments, fucosylation or fucosylated refers to fucose residues within the oligosaccharides attached to the peptide backbone of an antibody of the present disclosure. Specifically, a fucosylated antibody comprises α (1,6)-linked fucose at the innermost N-acetylglucosamine (GlcNAc) residue in one or both of the N-linked oligosaccharides attached to the antibody Fc region, e.g. at position Asn 297 of the human IgG1 Fc domain (EU numbering of Fc region residues). Asn297 may also be located about +3 amino acids upstream or downstream of position 297, i.e. between positions 294 and 300, due to minor sequence variations in immunoglobulins.

In some embodiments, a degree of fucosylation is a percentage of fucosylated oligosaccharides relative to all oligosaccharides, e.g., as identified by methods known in the art, such as in an N-glycosidase F treated antibody composition assessed by matrix-assisted laser desorption-ionization time-of-flight mass spectrometry (MALDI-TOF MS). In a composition of a fully fucosylated antibody, at least 90% or essentially all oligosaccharides comprise fucose residues, i.e. are fucosylated. Accordingly, an individual antibody in such a composition typically comprises fucose residues in each of the two N-linked oligosaccharides in the Fc region. In some embodiments, in a composition of a fully non-fucosylated antibody, less than about 10% or essentially none of the oligosaccharides are fucosylated, and an individual antibody in such a composition does not contain fucose residues in either of the two N-linked oligosaccharides in the Fc region. In a composition of a partially fucosylated antibody, only part of the oligosaccharides comprise fucose. An individual antibody in such a composition can comprise fucose residues in none, one or both of the N-linked oligosaccharides in the Fc region, provided that the composition does not comprise essentially all individual antibodies that lack fucose residues in the N-linked oligosaccharides in the Fc region, nor essentially all individual antibodies that contain fucose residues in both of the N-linked oligosaccharides in the Fc region. In one embodiment, a composition of a partially fucosylated antibody has a degree of fucosylation of about 10% to about 80% (e.g., about 50% to about 80%, about 60% to about 80%, or about 70% to about 80%).

In some embodiments, a glycosylation antibody variant comprises an Fc region, wherein a carbohydrate structure attached to the Fc region lacks fucose. Such variants have improved ADCC function. Examples of defucosylated or fucose-deficient antibodies are described in: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87: 614 (2004).

Antibodies with reduced fucosylation, or antibodies that are non-fucosylated may be produced using any method known in the art. In some embodiments of the antibodies of the disclosure, at least one or two of the heavy chains of the antibody can be non-fucosylated. For example, antibodies of the disclosure with reduced fucosylation, or antibodies of the disclosure that are non-fucosylated may be produced in a cell line having a alphal,6-fucosyltransferase (Fut8) knockout, and/or overexpressing β1,4-N-acetylglycosminyltransferase III (GnT-III) and/or overexpresses Golgi μ-mannosidase II (ManII) Antibodies with reduced fucosylation, or antibodies that are non-fucosylated may also be generated using a cell line that is deficient for ‘FUT8’, alpha-1,6 fucosyltransferase, which catalyzes the transfer of fucose; using Chinese hamster ovary (CHO) cells, e.g., that are deficient in FUT8 (Yamane-Ohnuki et al., 2004); using small interfering RNAs (siRNAs) to block the expression of the FUT8 gene (Mori et al., 2004). Other cell lines that may be used to produce non-fucosylated or defucosylated antibodies or antibodies with reduced fucosylation are known in the art, e.g., include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)), and cells overexpressing β1,4-N-acetylglycosminyltransferase III (GnT-III) and Golgi μ-mannosidase II (ManII)

In some embodiments, antibodies of the present disclosure have reduced fucose relative to the amount of fucose on the same antibody produced in a wild-type CHO cell. For example, an antibody can have a lower amount of fucose than it would otherwise have if produced by native CHO cells (e.g., a CHO cell that produce a native glycosylation pattern, such as, a CHO cell containing a native FUT8 gene). In some embodiments, an antibody provided herein is one wherein less than about 50%, 40%, 30%, 20%, 10%, 5% or 1% of the N-linked glycans thereon comprise fucose. In certain embodiments, an antibody provided herein is one wherein none of the N-linked glycans thereon comprise fucose, i.e., wherein the antibody is completely without fucose, or has no fucose or is non-fucosylated or is afucosylated. The amount of fucose can be determined by one of skill in the art, e.g., by calculating the average amount of fucose within the sugar chain at Asn297, relative to the sum of all glycostructures attached to Asn297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. In some embodiments, at least one or two of the heavy chains of the antibody is non-fucosylated.

Antibodies lacking 1,6-fucose on their heavy chain glycosylation may have enhanced binding affinity to the FcγRIII receptor and increased ADCC activity (see, e.g., Shields et al., 2002; Shinkawa et al, 2002; Okazaki, 2004; Dall'Ozzo, 2004). In some embodiments, the antibodies provided herein include an Fc region with modifications including reduced fucosylation, non-fucosylation, and/or mutations that enhance ADCC activities and/or improve affinity of the Fc region for Fc receptors such as FcγRIII and CD16. In some embodiments, the molecules (e.g., the antibodies provide herein) could induce antibody directed cell cytotoxicity (ADCC) and deplete or reduce the number of LGL and NK cells to a higher extent over a fucosylated or wild type antibody.

In some embodiments, an antibody of the disclosure is engineered to improve ADCC activity by reducing fucosylation. In some embodiments, the molecules provided herein (e.g., the antibodies provided herein) can induce antibody directed cell cytotoxicity (ADCC) and deplete or reduce number of LGL and/or NK cells to a higher extent than a fucosylated or wild type antibody. In some embodiments, at least one or two of the heavy chains of an antibody of the disclosure are non-fucosylated. In some embodiments, an antibody of the disclosure is modified such that the carbohydrates of the antibody are non-fucosylated. In some embodiments, an antibody of the disclosure is modified such that less than about 90%, e.g., less than any of about 90%, about 80%, about 70%, about 60%, about 50%, about 40%, about 30%, about 20%, about 10%, about 5%, or about 1%, of the carbohydrates of the antibody contain fucose. In some embodiments, an antibody of the disclosure is modified such that less than about 40% of the carbohydrates of the antibody contain fucose. In some embodiments, the antibodies provided herein are non-fucosylated.

In some embodiments, the molecules (e.g., antibodies) provided herein could induce antibody directed cell cytotoxicity (ADCC) and deplete or reduce the number of LGL and NK cells to a higher extent over a fucosylated or wild type antibody.

(iii) Mutations that Enhance ADCC Activity

An antibody of the disclosure may comprise a variant Fc region. In some embodiments, the variant Fc region includes at least one amino acid substitution in the Fc region that improves ADCC activity. For example, an antibody of the disclosure may have a variant IgG1 Fc region which comprises one or more of the Fc mutations selected from S239D, A330L, 1332E, F243L and G236A. In another example, an antibody of the disclosure may have a human IgG1 Fc variant region which comprises one or more of the Fc mutations selected from S239D, A330L, 1332E, F243L and G236A. Other amino acid substitutions that are known to enhance ADCC activity may be used, for example, as described in Lazar et al., PNAS 103, 4005-4010 (2006); Shields et al., J. Biol. Chem. 276, 6591-6604 (2001); Stewart et al., Protein Engineering, Design and Selection 24, 671-678 (2011), and Richards et al., Mol Cancer Ther 7, 2517-2527 (2008).

(iv) Reduced Internalization

In some embodiments, an antibody of the disclosure has a low degree of receptor-induced internalization, e.g., as compared to a wild type control antibody, or an antibody known in the art or commercially available for the same target. Antibodies with lower internalization have a higher receptor occupancy on the cell surface and higher level of the receptor-antibody complexes on the cell surface, which may enhance ADCC activity. An antibody of the disclosure may be tested in vitro for its target (e.g., any of CD94, CD57, or NKG2A) internalization capabilities. Antibody candidates with no or low internalization activity may be further tested for binding to a target from cynomolgus monkeys and/or from humans (e.g., any of cynomolgus and/or human CD94, cynomolgus and/or human CD57, or cynomolgus and/or human NKG2A). Antibodies that bind to a cynomolgus and/or human target may be used for cell killing assays (e.g., ADCC assays) in vitro and in vivo. The cell killing activity (e.g., ADCC activity) of the selected antibodies may be compared to the commercially available antibodies or antibodies known in the art.

B. Generation of Antibodies

An antibody of the disclosure may be generated using any technologies and/or methods known in the art. Techniques for preparing antibodies, e.g., monoclonal antibodies (mAbs), against virtually any target antigen are well known in the art. See, for example, Köhler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991). Briefly, monoclonal antibodies can be obtained by injecting mice with a composition comprising an antigen (e.g., any of CD94, CD57, or NKG2A, or a part thereof), removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures. The person of ordinary skill will realize that where antibodies are to be administered to human subjects, the antibodies will bind to human antigens (e.g., any of human CD94, human CD57, or human NKG2A, or a part thereof).

MAbs can be isolated and purified from hybridoma cultures by a variety of well-established techniques. Such isolation techniques include affinity chromatography with Protein-A or Protein-G Sepharose, size-exclusion chromatography, and ion-exchange chromatography. See, for example, Coligan at pages 2.7.1-2.7.12 and pages 2.9.1-2.9.3. Also, see Baines et al., “Purification of Immunoglobulin G (IgG),” in METHODS IN MOLECULAR BIOLOGY, VOL. 10, pages 79-104 (The Humana Press, Inc. 1992).

After the initial raising of antibodies to the immunogen (e.g., any of CD94, CD57, or NKG2A, or a part thereof), the antibodies can be sequenced and subsequently prepared by recombinant techniques. Humanization and chimerization of murine antibodies and antibody fragments are well known to those skilled in the art, as discussed below.

In an exemplary method of generating an antibody of the disclosure, recombinant targets (e.g., any of CD94, CD57, or NKG2A) may be utilized for immunization of mice. Antibodies generated following immunization of mice, e.g., as described above, may be analyzed for specific or selective binding to its target (e.g., any of CD94, CD57, or NKG2A) by ELISA and flow cytometry. Antibodies may be selected based on their ability to bind to a target (e.g., any of CD94, CD57, or NKG2A).

In some embodiments, non-human primate antibodies may be generated. General techniques for raising therapeutically useful antibodies in baboons may be found, for example, in Goldenberg et al., WO 91/11465 (1991), and in Losman et al., Int. J. Cancer 46: 310 (1990).

In some embodiments, an antibody may be a human antibody. In some embodiments, an antibody may be a monoclonal human antibody. In some embodiments, a human antibody possesses an amino acid sequence which corresponds to that of an antibody produced by a human or a human cell or derived from a non-human source that utilizes human antibody repertoires or other human antibody-encoding sequences. Such antibodies may be obtained from transgenic mice that have been engineered to produce specific human antibodies in response to antigenic challenge e.g., any of CD94, CD57, or NKG2A, or a part thereof. Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Phamacol. 3:544-50). In certain embodiments, the claimed methods and procedures may utilize human antibodies produced by such techniques. Other methods of producing fully human antibodies include phage display, e.g., as described in Dantas-Barbosa et al., 2005, Genet. Mol. Res. 4:126-40, generation of antibodies in normal humans or from humans that exhibit a particular disease state, e.g., as described in Dantas-Barbosa et al., 2005, or using transgenic animals (e.g., mice) that have been genetically engineered to produce human antibodies using standard immunization protocols as discussed above, e.g., as described in Green et al., 1999, J. Immunol. Methods 231:11-23, Green et al., Nature Genet. 7:13 (1994), Lonberg et al., Nature 368:856 (1994), and Taylor et al., Int. Immun. 6:579 (1994).

(i) In Vitro Cell Killing Assays

Generation of an antibody of the disclosure may involve testing the in vitro ADCC activity of the antibody. The improved cell killing or ADCC activity of an antibody of the disclosure (e.g., an antibody that is non-fucosylated, and/or includes mutations or amino acid substitutions that enhance ADCC activity, e.g., a variant or mutated Fc region, and/or has a low level of internalization) may be tested for depletion of LGL and/or NK cells. Depletion of LGL and/or NK cells may be tested using an exemplary in vitro model that recapitulates activity in humans (Tomasevic, et al, Growth Factors, 2014; 32(6): 223-235; Huang, et al, JCI insight, 2016; 1(7):e86689). Peripheral blood lymphocytes (PBL) isolated from the blood of normal (i.e., healthy) donors are incubated with antibodies that have a human Fc region with and without fucose and/or with and without Fc region mutations. The level of killing of LGL or NK cells in the PBLs (e.g., in a PBL sample) is measured using any method known in the art, such as flow cytometry (e.g., as described in the Examples). The cell killing activity (e.g., ADCC activity) of antibodies may be tested as described above, e.g., using the assay described above, using a variety of biospecimens such as blood, synovial fluid, bone marrow and spleen intact cell homogenates from patients with diseases such as chronic lymphoproliferative disorder of NK cells (CLPD-NK), LGL leukemia, Felty's syndrome, IBM and RA with LGL and/or aggressive NK leukemia.

In addition to the cell killing assay described above, in vitro ADCC and antibody-dependent cellular phagocytosis (ADCP) assays using antibodies of the disclosure, e.g., selected candidate antibodies of the disclosure, purified target cells (e.g., LGL or NK cells) and/or effector cells such as NK cells or monocytes/macrophages may be performed to assay the cell killing, ADCC and/or ADCP activity of antibodies of the disclosure. Cell killing, ADCC and/or ADCP assays, and other assay methods known in the art may be used, for example, as described in Kolbeck et al., J Allergy Clin Immunol. 2010; 125(6):1344-1353.e2; Gomez-Roman et al., J. Immunol. Methods, 2006, 308, pp. 53-67; and Ackerman et al., J. Immunol. Methods, 2011, 366, pp. 8-19. The in vitro activity of an antibody of the disclosure may be compared to a commercially available antibody or an antibody known in the art against the same target.

(ii) In Vivo Cell Killing Assays

Generation of an antibody of the disclosure may involve testing the in vivo ADCC activity of the antibody, e.g., to show activity of the selected antibody candidates in vivo for depletion or reduction in the levels of LGL or NK cells. The in vivo cell killing activity (e.g., ADCC and/or ADCP activity) of an antibody of the disclosure may be determined using any method known in the art. For example, the ability of an antibody of the disclosure to deplete or reduce LGL or NK cells in vivo may be tested in cynomolgus monkeys using methods known in the art. For example, in an exemplary method to test the in vivo cell killing activity (e.g., ADCC and/or ADCP activity) of an antibody of the disclosure, a cohort of cynomolgus monkeys are bled one day prior to administration of a single dose of an antibody of the disclosure, e.g., antibody treatment, to identify the pre-dose levels of LGL and NK cells by flow cytometry. After administration of an antibody of the disclosure, e.g., upon treatment with antibodies of the disclosure, the monkeys are bled at the following time points: 1 hour, 1 day, 7 days, 14 days and 30 days. The levels of LGL and NK cells in blood and other biospecimens such as synovial fluids, bone marrow and spleen are determined by flow cytometry at each of the time points. The in vivo activity of an antibody of the disclosure may be compared to a commercially available antibody or an antibody known in the art against the same target. For example, an anti-CD94 antibody of the disclosure, e.g., an anti-CD94 mAb candidate, may be compared to anti-CD94 antibody DX22, a commercially available anti-CD94 mAb that has been reported to cross-react with cynomolgus CD94. A skilled artisan will readily appreciate that other methods known in the art for testing ADCC activity in vivo may be used to assay the in vivo ADCC activity of antibodies of the disclosure (e.g., transgenic animals such as transgenic mice).

Other known antibodies against the targets (e.g., any of CD94, CD57, or NKG2A) may also be used in the methods provided herein. For example, an anti-CD94 mAb of the disclosure may be tested (e.g., for in vitro or in vivo ADCC activity, or for any other characteristic described herein) together with the following anti-CD94 antibodies: HP-3D9 (LSBio Catalog # LS-C134679-100; Abnova Catalog #: MAB6947); 212; 131412 (R&D Systems Catalog #: MAB1058); 13B146 (US Biological Catalog #: 030068); 13B147 (US Biological Catalog #: 030069); 1H1 (Abnova Catalog #: MAB10543); 3G2 (Biorbyt Catalog #: orb69389); DX22 (Biolegend Catalog # 305502); REA113 (Miltenyi Biotec Catalog #: 130-098-967); KP43; EPR21003; AT13E3 (ATGen Catalog: ATGA0487) and B-D49.

(iii) Humanization

An antibody of the disclosure may be humanized according to any method known in the art. In some embodiments, a humanized antibody is a chimeric antibody comprising amino acid residues from non-human hypervariable regions (HVRs) and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. In some embodiments, a humanized form of an antibody, e.g., a non-human antibody, refers to an antibody that has undergone humanization.

For example, a monoclonal antibody may be humanized by transferring mouse CDRs from the heavy and light variable chains of a mouse immunoglobulin into the corresponding variable domains of a human antibody. The mouse framework regions (FR) in the chimeric monoclonal antibody may also be replaced with human FR sequences. To preserve the stability and antigen specificity of the humanized monoclonal antibody, one or more human FR residues may be replaced by the mouse counterpart residues Humanized monoclonal antibodies may be used for therapeutic treatment of subjects. Techniques for the production of humanized monoclonal antibodies are well known in the art, e.g., as described in Jones et al., 1986, Nature, 321:522; Riechmann et al., Nature, 1988, 332:323; Verhoeyen et al., 1988, Science, 239:1534; Carter et al., 1992, Proc. Nat'l Acad. Sci. USA, 89:4285; Sandhu, Crit. Rev. Biotech., 1992, 12:437; Tempest et al., 1991, Biotechnology 9:266; Singer et al., J. Immun., 1993, 150:2844.

(iv) Selection

An antibody of the disclosure may be selected based on parameters described above, such as enhanced in vitro and/or in vivo cell killing activity (e.g., ADCC and/or ADCP activity), enhanced binding to one or more Fc receptors, level of fucosylation (e.g., reduced fucosylation, or non-fucosylation), and/or affinity for its target protein (e.g., any of CD94, CD57, or NKG2A).

In some embodiments, an antibody of the disclosure, e.g., a humanized antibody of the disclosure, may be selected based on its binding characteristics (e.g., affinity) to human and/or cynomolgus monkey CD94, CD57, or NKG2A. In some embodiments, an antibody of the disclosure, e.g., a humanized antibody of the disclosure, may be selected based on its internalization ability, e.g., as described above. In some embodiments, an antibody of the disclosure, e.g., a humanized antibody of the disclosure, may be selected based on its cell killing, ADCC and/or ADCP activities in vivo and/or in vitro, e.g., as described above.

In some embodiments, an antibody of the disclosure, e.g., a humanized antibody of the disclosure may be selected based on its solubility. In some embodiments, an antibody of the disclosure is selected if it is soluble at concentrations higher than about 10 mg/mL. In some embodiments, an antibody of the disclosure, e.g., a humanized antibody of the disclosure may be selected based on the level soluble aggregates formed in a solution of the antibody. For example, an antibody of the disclosure is selected if it has low level of soluble aggregates (e.g., less than 5%, less than 4%, less than 3%, less than 2%, or less than 1% soluble aggregates). In some embodiments, an antibody of the disclosure, e.g., a humanized antibody of the disclosure may be selected based on its ability to maintain binding to its target (e.g., any of CD94, CD57, or NKG2A) during storage, e.g., for at least 1 week, at least 2 weeks, at least 3 weeks, at least 1 month, at least 2 months, at least 3 months, at least 4 months, or more, at any of about 2° C., about 3° C., about 4° C., about 5° C., about 6° C., about 7° C., about 8° C. In some embodiments, an antibody of the disclosure, e.g., a humanized antibody of the disclosure may be selected based on its stability (e.g., lack of degradation products, e.g., as measured by SDS-PAGE) during storage, e.g., for at least 1 week, at least 2 weeks, at least 3 weeks, at least 1 month, at least 2 months, at least 3 months, at least 4 months, or more, at any of about 2° C., about 3° C., about 4° C., about 5° C., about 6° C., about 7° C., or about 8° C.

In some embodiments, an antibody of the disclosure, e.g., a humanized antibody of the disclosure may be selected based on its toxicology. Toxicology analysis of an antibody of the disclosure may be carried out using any method known in the art. In an exemplary toxicology analysis, an antibody of the disclosure, e.g., a humanized antibody of the disclosure, is tested for toxicity in cynomolgus monkeys at doses that are more than 5 times higher (e.g., any of about 5 times higher, about 10 times higher, about 15 times higher, about 20 times higher, about 25 times higher, about 30 times higher, about 35 times higher, about 40 times higher, about 45 times higher, about 50 times higher, about 55 times higher, about 60 times higher, about 65 times higher, about 70 times higher, about 75 times higher, about 80 times higher, about 85 times higher, about 90 times higher, about 95 times higher, about 100 times higher, or more) than the doses anticipated to be used in human subjects.

In some embodiments, an antibody of the disclosure, e.g., a humanized antibody of the disclosure may be selected based on its ability to deplete or reduce level of LGL and/or NK cells in vitro and/or in vivo. Depletion or reduction in the level of LGL and/or NK cells may be measured using any method known in the art. For example, depletion or of LGL and/or NK cells may be measured using a cell killing, ADCC, and/or ADCP assay, e.g., as described above and/or as described in the Examples. In some embodiments, the final mAb candidate can be humanized and characterized for binding to human and cynomolgus CD94 or CD57 or NKG2a, internalization and ADCC abilities, and in vivo activity.

In addition, in some embodiments, the final candidate needs to be soluble at concentrations higher than 10mg/mL, has low level of soluble aggregates (<5%), maintains its binding to the target as measured by ELISA (>90% potency), with no degradation products as measured by SDS PAGE when incubated for 3 months at 2-8° C. In some embodiments, toxicology analysis of the final humanized candidate can be performed in cynomolgus monkeys at doses that are more than 5 times higher than the doses anticipated to be used in human subjects.

In some embodiments, the antibodies that bind to CD94 or CD57 or NKG2A may deplete or reduce level of LGL or NK cells and may have clear benefits for patients (e.g., human patient) such as LGL leukemia, Rheumatoid arthritis, Felty's syndrome, aggressive NK leukemia, IBM, IBD etc. In addition, the antibody treatment may have better tolerability and fewer side-effects over the first and second line of therapies including chemo, chemotherapy, Alemtuzumab and splenectomy. The antibody treatment may demonstrate more selective depletion of the disease inducing cells compared to the current therapies that are non-selective. Nonlimiting examples of diseases and disorders in which LGL and NK cells play a role are: LGL leukemia, Rheumatoid arthritis, Felty's syndrome, aggressive NK leukemia, IBM, IBD etc. Accordingly, the invention provides a method of reducing the number or depleting of LGL or NK cells in a human subject upon administration of molecule that binds to cell surface protein on LGL or NK cells such as CD94 or CD57or NKG2A or an additional cell surface protein that is specific for LGL cells and that comprises (a) a region that specifically binds to the target and (b) an immunoglobulin Fc region.

C. Antibody Targets

An antibody of the disclosure may specifically bind to CD94, CD57, or NKG2A. Also encompassed by the disclosure are antibodies that bind to a cell surface protein that is expressed on LGL and/or NK cells. Techniques for preparing antibodies, e.g., monoclonal antibodies (mAbs), against virtually any target antigen are well known in the art, e.g., as described above. Accordingly, an antibody that binds to a cell surface protein that is expressed on LGL and/or NK cells may be used in any of the methods, compositions, articles of manufacture or kits disclosed herein.

In some embodiments, the terms bind, specifically binds to, or is specific for refer to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (K_{D}) of <1 μM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

In some embodiments, an antibody of the disclosure binds to human CD94, human CD57, or human NKG2A. In some embodiments, an antibody of the disclosure binds to cynomolgus CD94, cynomolgus CD57, or cynomolgus NKG2A. In some embodiments, an antibody of the disclosure binds to human and cynomolgus CD94, human and cynomolgus CD57, or human and cynomolgus NKG2A.

In some embodiments, the antibodies provided herein bind to human CD94 (Natural killer cells antigen CD94; CD94 Entrez Gene ID: 3824; KLRD1 (HGNC Symbol); UniProtKB identifier: Q13241; HGNC:6378; Ensembl: ENSG00000134539 OMIM: 602894; KP43), human CD57 (CD57; B3GAT1 (Beta-1,3-Glucuronyltransferase 1); LEU7; GLCUATP 3; GlcAT-P 4; HNK1, NK-1, NK1; HGNC: 921; Entrez Gene: 27087; Ensembl: ENSG00000109956 OMIM: 151290; UniProtKB: Q9P2W7) or human NKG2A (NKG2-A/NKG2-B type II integral membrane protein; Gene: KLRC1, UniProtKB: P26715 (NKG2A_HUMAN); CD159 antigen-like family member A; HGNC:6374).

In some embodiments, an antibody of the disclosure binds to a human CD94 protein or a part thereof, or a protein having at least 80% (e.g., any of at least 80%, at least 85%, at least 90%, at least 95%, at least 96%, at least 97%, at least 98%, at least 99%, or 100%) homology to a human CD94 protein or a part thereof Amino acid sequences of exemplary human CD94 proteins are provided in the sequences of SEQ ID NOs: 1-3:

(SEQ ID NO: 1)
MAVFKTTLWRLISGTLGIICLSLMSTLGILLKNSF
TKLSIEPAFTPGPNIELQKDSDCCSCQEKWVGYRC
NCYFISSEQKTWNESRHLCASQKSSLLQLQNTDEL
DFMSSSQQFYWIGLSYSEEHTAWLWENGSALSQYL
FPSFETFNTKNCIAYNPNGNALDESCEDKNRYICK
QQLI
(SEQ ID NO: 2)
MAVFKTTLWRLISGTLGIICLSLMSTLGILLKNSF
TKLSIEPAFTPGPNIELQKDSDCCSCQEKWVGYRC
NCYFISSEQKTWNESRHLCASQKSSLLQLQNTDEL
QDFMSSSQQFYWIGLSYSEEHTAWLWENGSALSQY
LFPSFETFNTKNCIAYNPNGNALDESCEDKNRYIC
KQQLI
(SEQ ID NO: 3)
MAAFTKLSIEPAFTPGPNIELQKDSDCCSCQEKWV
GYRCNCYFISSEQKTWNESRHLCASQKSSLLQLQN
TDELDFMSSSQQFYWIGLSYSEEHTAWLWENGSAL
SQYLFPSFETFNTKNCIAYNPNGNALDESCEDKNR
YICKQQLISYSEEHTAWLWENGSALSQYLFPSFET
FNTKNCIAYNPNGNALDESCEDKNRYICKQQLI

In some embodiments, an antibody of the disclosure binds to a human NKG2A protein or a part thereof, or a protein having at least 80% (e.g., any of at least 80%, at least 85%, at least 90%, at least 95%, at least 96%, at least 97%, at least 98%, at least 99%, or 100%) homology to a human NKG2A protein or a part thereof Amino acid sequences of exemplary human NKG2A proteins are provided in the sequences of SEQ ID NOs: 4 & 5:

(SEQ ID NO: 4)
MDNQGVIYSDLNLPPNPKRQQRKPKGNKNSILATE
QEITYAELNLQKASQDFQGNDKTYHCKDLPSAPEK
LIVGILGIICLILMASVVTIVVIPSTLIQRHNNSS
LNTRTQKARHCGHCPEEWITYSNSCYYIGKERRTW
EESLLACTSKNSSLLSIDNEEEMKFLSIISPSSWI
GVFRNSSHHPWVTMNGLAFKHEIKDSDNAELNCAV
LQVNRLKSAQCGSSIIYHCKHKL
(SEQ ID NO: 5)
MDNQGVIYSDLNLPPNPKRQQRKPKGNKNSILATE
QEITYAELNLQKASQDFQGNDKTYHCKDLPSAPEK
LIVGILGIICLILMASVVTIVVIPSRHCGHCPEEW
ITYSNSCYYIGKERRTWEESLLACTSKNSSLLSID
NEEEMKFLSIISPSSWIGVFRNSSHHPWVTMNGLA
FKHEIKDSDNAELNCAVLQVNRLKSAQCGSSIIYH
CKHKL

In some embodiments, an antibody of the disclosure binds to a human CD57 protein or a part thereof, or a protein having at least 80% (e.g., any of at least 80%, at least 85%, at least 90%, at least 95%, at least 96%, at least 97%, at least 98%, at least 99%, or 100%) homology to a human CD57 protein or a part thereof Amino acid sequences of exemplary human CD57 proteins are provided in the sequences of SEQ ID NOs: 6 & 7:

(SEQ ID NO: 6)
MTDSVIYSMLELPTATQAQNDYGPQQKSSSSRPSC
SCLVAIALGLLTAVLLSVLLYQWILCQGSNYSTCA
SCPSCPDRWMKYGNHCYYFSVEEKDWNSSLEFCLA
RDSHLLVITDNQEMSLLQVFLSEAFCWIGLRNNSG
WRWEDGSPLNFSRISSNSFVQTCGAINKNGLQASS
CEVPLHWVCKKCPFADQALF
(SEQ ID NO: 7)
MTDSVIYSMLELPTATQAQNDYGPQQKSSSSRPSC
SCLVAIALGLLTAVLLSVLLYQWILCQGSNYSTCA
SCPSCPDRWMKYGNHCYYFSVEEKDWNSSLEFCLA
RDSHLLVITDNQEMSLLQVFLSEAFCWIGLRNNSG
WRWEDGSPLNFSRISSNSFVQTCGAINKNGLQASS
CEVPLHWVCKKVRL

In some embodiments, an antibody of the disclosure binds to its target (e.g., any of CD94, CD57, or NKG2A) in the same or a different epitope as an antibody known in the art for that target. In some embodiments, an antibody of the disclosure binds to a different epitope as an antibody known in the art. In some embodiments, an antibody of the disclosure specifically binds to human CD94, wherein the antibody does not bind to the same epitope on human CD94 as anti-CD94 antibody clones HP-3D9, DX22, 131412, or 12K45. In some embodiments, an antibody of the disclosure specifically binds to human CD57, wherein the antibody does not bind to the same epitope on human CD57 as anti-CD57 antibody clone NK-1. In some embodiments, an antibody of the disclosure specifically binds to human NKG2A, wherein the antibody does not bind to the same epitope on human NKG2A as anti-NKG2A antibody clone Z199.

In some embodiments, if an antibody of the disclosure does not bind to its target (e.g., any of CD94, CD57, or NKG2A) in the same epitope as another antibody for that target, e.g., a commercially available antibody or an antibody known in the art for that target, then the antibody of the disclosure does not block binding of the other antibody to the target in a competition assay, e.g., by 50% or more. In some embodiments, if an antibody of the disclosure does not bind to its target (e.g., any of CD94, CD57, or NKG2A) in the same epitope as another antibody for that target, e.g., a commercially available antibody or an antibody known in the art for that target, then the other antibody does not block binding of the antibody of the disclosure to the target in a competition assay, e.g., by 50% or more.

In some embodiments, an antibody of the disclosure binds to its target (e.g., any of CD94, CD57, or NKG2A) with a higher affinity than an antibody known in the art for that target. In certain embodiments, the affinity of an antibody for its target (e.g., any of CD94, CD57, or NKG2A) may be represented by the dissociation constant (K_{D}). Affinity can be measured by common methods known in the art, such as flow cytometry or Western blotting. In some embodiments, the K_{D} is measured using a radiolabeled antigen binding assay (RIA) performed with the Fab version of an antibody of the disclosure and its target (e.g., any of CD94, CD57, or NKG2A). In some embodiments, the K_{D} is measured using surface plasmon resonance assays. Exemplary assays are described, e.g., in Drake, A. W. and Klakamp, S. L. (2007) J. Immunol. Methods 318:147-152.

In some embodiments, an antibody of the disclosure specifically binds to human CD94, wherein the antibody binds to human CD94 with a greater affinity than anti-CD94 antibody clones HP-3D9, DX22, 131412, and 12K45. In some embodiments, an antibody of the disclosure specifically binds to human CD57, wherein the antibody binds to human CD57 with greater affinity than anti-CD57 antibody clone NK-1. In some embodiments, an antibody of the disclosure specifically binds to human NKG2A, wherein the antibody binds to human NKG2A with a greater affinity than anti-NKG2A antibody clone Z199. In some embodiments, an antibody of the disclosure binds to its target (e.g., any of CD94, CD57, or NKG2A) with any of at least 1.5-fold, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, at least 10-fold, or more, greater affinity than another antibody known in the art for that target.

In certain embodiments, an antibody of the disclosure has a K_{D} of less than about 10 μM for binding to its target (e.g., any of CD94, CD57, or NKG2A). In certain embodiments, an antibody of the disclosure has a K_{D} of less than about 1 μM for binding to its target (e.g., any of CD94, CD57, or NKG2A). In certain embodiments, an antibody of the disclosure has a K_{D} of any of less than about 1000 nM, less than about 900 nM, less than about 800 nM, less than about 700 nM, less than about 600 nM, less than about 500 nM, less than about 400 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 9 nM, less than about 8 nM, less than about 7 nM, less than about 6 nM, less than about 5 nM, less than about 4 nM, less than about 3 nM, less than about 2 nM, less than about 1 nM, less than about 0.5 nM, or less than about 0.1 nM for binding to its target (e.g., any of CD94, CD57, or NKG2A). In some embodiments, an antibody of the disclosure has a K_{D} of any of less than about 100 pM, less than about 75 pM, less than about 50 pM, less than about 25 pM, less than about 10 pM, less than about 5 pM, less than about 1 pM, less than about 0.5 pM, or less than about 0.1 pM for binding to its target (e.g., any of CD94, CD57, or NKG2A).

Other known antibodies against the targets (e.g., any of CD94, CD57, or NKG2A) may also be used in the methods provided herein. For example, the following anti-CD94 antibodies may be used: HP-3D9 (LSBio Catalog # LS-C134679-100; Abnova Catalog #: MAB6947); 212; 131412 (R&D Systems Catalog #: MAB1058); 13B146 (US Biological Catalog #: 030068); 13B147 (US Biological Catalog #: 030069); 1H1 (Abnova Catalog #: MAB10543); 3G2 (Biorbyt Catalog #: orb69389); DX22 (Biolegend Catalog # 305502); REA113 (Miltenyi Biotec Catalog #: 130-098-967); KP43; EPR21003; AT13E3 (ATGen Catalog: ATGA0487) and B-D49.

### III. Pharmaceutical Formulations

In some embodiments, a pharmaceutical composition, a composition, or a pharmaceutical formulation refer to a biologically active compound (e.g., an antibody of the disclosure), optionally mixed with at least one pharmaceutically acceptable chemical component, such as, though not limited to carriers, stabilizers, diluents, dispersing agents, suspending agents, thickening agents, excipients and the like.

Pharmaceutical compositions, pharmaceutical formulations, and/or compositions of any of the antibodies of the disclosure for use in any of the methods as described herein may be prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The formulation herein may also contain more than one active ingredient as necessary for the particular indication (e.g., a disease or disorder) being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or immunoconjugate, which matrices are in the form of shaped articles, e.g., films, or microcapsules.

The formulations to be used for in vivo administration are generally sterile. Sterility may be readily accomplished, e.g., by filtration through sterile filtration membranes.

### IV. Kits and Articles of Manufacture

In another aspect of the disclosure, a kit or an article of manufacture containing materials useful for the methods provided herein, e.g., treatment the disease or disorders described above, reducing the number of peripheral blood LGL and/or NK cells in a subject, or inducing ADCC activity in a subject, are provided. The kit or article of manufacture may comprise a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for the methods provided herein, e.g., treatment of the disease or disorders described above, reducing the number of peripheral blood LGL and/or NK cells in a subject, or inducing ADCC activity in a subject, and may have a sterile access port (for example the container may be an intravenous solution bag or a vial having a stopper pierceable by a hypodermic injection needle). At least one active agent in the composition is an antibody of the disclosure. The label or package insert indicates that the composition is used for the methods provided herein, e.g., treatment of the disease or disorders described above, reducing the number of peripheral blood LGL and/or NK cells in a subject, or inducing ADCC activity in a subject. Moreover, the kit or article of manufacture may comprise (a) a first container with a composition contained therein, wherein the composition comprises an antibody of the disclosure; and (b) a second container with a composition contained therein, wherein the composition comprises a further therapeutic agent. The kit or article of manufacture in this embodiment of the invention may further comprise a package insert indicating that the compositions can be used to treat a particular disease or disorder, e.g., as described herein, to reduce the number of peripheral blood LGL and/or NK cells in a subject, or to induce ADCC activity in a subject. Alternatively, or additionally, the kit or article of manufacture may further comprise a second (or third) container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1

### Analysis of CD94 Expression on Immune Cells from Healthy Donors and from T-large Granular Lymphocyte Leukemia (T-LGLL) and Chronic Lymphoproliferative Disorder of NK Cells (CLPD-NK) Patients

This Example describes the results of experiments to determine the level of CD94 receptor expression on immune cells obtained from healthy donors and from patients with T-LGLL and NK-LGLL.

### Materials and Methods

Healthy Donors and Patient Samples

Fresh healthy donor buffy coats were obtained from Stanford Blood Center. Peripheral blood mononuclear cells (PBMCs) were isolated via ficoll-paque (GE Healthcare, Chicago, Ill.) separation and cryopreserved in Bambanker cell freezing media (Bulldog-Bio, Portsmouth, N.H.). Briefly, buffy coats were diluted in phosphate buffered saline (PBS) in a 1:1 ratio, followed by layering of the diluted buffy coat and centrifugation at 760 g in ficoll. The PBMC layer was isolated and washed in PBS prior to downstream analysis. Peripheral blood leukocytes (PBLs) were isolated through red blood cell lysis. Frozen patient LGLL PBMCs were obtained. Tissue samples were provided by the Cooperative Human Tissue Network. Tissue dissociation was performed using the Miltenyi Biotec tumor dissociation kit according to the manufacturer's instructions.

Flow Cytometry Analysis

Approximately 1×10⁵-5×10⁵ cells were plated in non-tissue culture treated, 96-well V bottom plates and incubated in human FcgR blocking antibody (Biolegend, San Diego, Calif.) for 10 minutes at room temperature. The cells were subsequently stained with the eFluor 506 viability dye (ThermoFisher, Waltham, Mass.) in a 1:1000 dilution for 30 minutes on ice, followed by a wash step in FACS buffer (PBS with 2% fetal bovine serum). Antibody cocktail was added to the cells and incubated on ice for 30 minutes, followed by an additional wash step in FACS buffer. Ultracomp beads (ThermoFisher, Waltham, Mass.) were used for antibody compensation. The antibodies used in this study are provided in Table 1.

All data acquisition and fluorescence compensation were performed using CytoFlex (Beckman Coulter, Atlanta, Ga.). Data analysis was performed using FlowJo software. Single cells were gated using forward scatter area and forward scatter height, followed by live cell gating using eFluor 506 and forward scatter area. Monocytes were gated using a CD14+ strategy; T cells were gated using a CD3+/CD4+/CD8+ strategy; B cells were gated using a CD3-CD19+ strategy; NK cells were gated using a CD3−/CD56+/CD57+/CD16+ strategy; T-LGL leukemic cells were gated using a CD3+CD16+ strategy; NK-LGL leukemic cells were gated using a CD3-CD16+ strategy; and epithelial cells were identified using a CD45− strategy. Granulocytes were gated individually on forward and side scatter.

CD94, CD56 and NKG2A expression was determined on individual immune cell types using the markers described above.

Receptor Quantification

CD94, CD56 and NKG2A receptor numbers were quantified by staining PBMCs with APC-conjugated anti-target antibodies and gated based on the appropriate immune cell types (e.g., as described above). Quantum APC molecules of equivalent soluble fluorochrome (MESF) calibration standard beads (Bangs Laboratories, Inc., Fishers, Ind.) were analyzed concurrently to allow conversion of median fluorescence intensity (MFI) measurements to MESF units, according to the manufacturer's protocol. Background fluorescence was removed by subtracting the FMO (fluorescence minus one) and isotype control MESF values. MESF values were subsequently divided by the fluorophore to protein ratio (provided by the manufacturer) to convert to antibody binding capacity or receptor number.

Antibodies

Table 1 provides the antibodies used in the experiments described in Examples 1-3.

TABLE 1
Fluorescently-labeled antibodies.

| Target | Clone | Fluorophore | Catalog number | Vendor | Dilution |
|---|---|---|---|---|---|
| CD94 | HP-3D9 | APC | 559876 | BD Bioscience | 1:10 |
| CD94 | DX22 | APC | 305508 | Biolegend | 1:40 |
| CD94 | 131412 | APC | FAB1058A | R&D | 1:33 |
| CD94 | 12K45 | APC | C2442-09G | US Biologicals | 1:40 |
| NKG2A | Z199 | APC | A60797 | Beckman Coulter | 1:25 |
| CD14 | HCD14 | PE-Cy7 | 368606 | Biolegend | 1:20 |
| CD3 | SK7 | Pacific blue | 344824 | Biolegend | 1:20 |
| CD4 | OKT4 | Alexa Fluor 700 | 317426 | Biolegend | 1:40 |
| CD8 | SK1 | PerCP-Cy5.5 | 344710 | Biolegend | 1:20 |
| CD19 | B4 | Brilliant violet 605 | 302244 | Biolegend | 1:67 |
| CD16 | 3G8 | APC-Cy7 | 557758 | BD Bioscience | 1:200 |
| CD56 | B159 | FITC | 562794 | BD Bioscience | 1:67 |
| CD57 | NK-1 | PE | 560844 | BD Bioscience | 1:200 |
| CD45 | HI30 | PE | 304058 | Biolegend | 1:40 |
| mIgG1 | MOPC-21 | APC | 400120 | Biolegend | — |
| mIgG2a | MOPC-21 | APC | 981906 | Biolegend | — |
| mIgG2b | 27-35 | APC | 402206 | Biolegend | — |
| mIgM | MM-30 | PE | 401609 | Biolegend | — |

### Results

Healthy Donors

PBMC samples from six healthy donors were used to screen for CD94 expression. An additional three PBMC samples and two peripheral blood leukocyte (PBL) samples from healthy donors were used to screen for CD57 and NKG2A expression. Target expression on granulocytes was analyzed in two PBL samples. As shown in Table 2, CD94 was heavily expressed on NK cells (>50%), as indicated by staining with all four anti-CD94 commercial antibody clones. CD94 expression was low or not detected on monocytes, CD3+CD4+ T cells and B cells. A small subset of CD3+CD8+ T cells expressed CD94 (approximately 10-30%). NK cells (CD3-CD56+ and CD3-CD16+) expressed CD57 and NKG2A in the range of 60-70% and 40-45%, respectively. 15% of CD3+CD4+ and 45% of CD3+CD8+ T cells also expressed CD57. Granulocytes did not express any of the targets. All target expression results recapitulated results reported in the literature (see, e.g., Loughran T P J. (1993) Blood, 82(1):1-14; Zambello R (2014) Tansl Med UniSa, 8:4-11). Overall, these results showed that CD94 is selectively expressed on NK cells and subsets of CD3+CD8+ T cells.

TABLE 2
Flow cytometry analysis of CD94, CD57 and NKG2A in six healthy donor PBMC and PBL
samples. The values represent the percent cells positive for the indicated markers, with the range among
the donor PBMC and PBL samples in brackets.

| | | Monocytes | Granulocytes | T cells | | B cells | NK cells | | |
|---|---|---|---|---|---|---|---|---|---|
| Target | Ab Clone | CD14 | FSC/SSC | CD3+CD4+ | CD3+CD8+ | CD3−CD19+ | CD3−CD56+ | CD3−CD57+ | CD3−CD16+ |
| CD94 | HP-3D9 | Negative | Negative | Negative | 24 | Negative | 76.8 | 72.8 | 77.2 |
| | | | | | (15.9-39.5) | | (63.4-85.9) | (63.5-85.3) | (68.3-81.3) |
| CD94 | DX22 | Negative | Negative | Negative | 33.5 | Negative | 89.3 | 83.6 | 87.2 |
| | | | | | (23-50.1) | | (87.2-93.5) | (73.8-94.6) | (82.1-92.2) |
| CD94 | 131412 | Negative | Negative | Negative | 11.8 | Negative | 55.0 | 50.1 | 50.9 |
| | | | | | (5.39-22.5) | | (43.5-61) | (37.6-63.4) | (40.9-59.1) |
| CD94 | 12K45 | Negative | Negative | Negative | 10.3 | Negative | 57.3 | 46.9 | 47.3 |
| | | | | | (3.97-14.3) | | (49.5-67.1) | (33.8-53.8) | (36.7-54) |
| CD-57 | NK-1 | Negative | Negative | 14.4 | 43.2 | Negative | 66.5 | 100 | 73.0 |
| | | | | (12.5-16.8) | (22.2-58.3) | | (42.5-74.8) | | (56.4-85.1) |
| NKG2A | Z199 | Negative | Negative | Negative | 10.2 | Negative | 44.9 | 40.5 | 40.0 |
| | | | | | (3.7-19.7) | | (34.3-59.3) | (22.2-57.4) | (29-55.6) |

Blood samples from healthy donors were also analyzed to determine the number of CD94 receptors on CD14+ monocytes, CD3+CD4+ and CD3+CD8+ T cells, CD3-CD19+ B cells, granulocytes (based on FSC/SSC) and CD3-CD57+, CD3-CD16+ and CD3-/CD56+ NK cells.

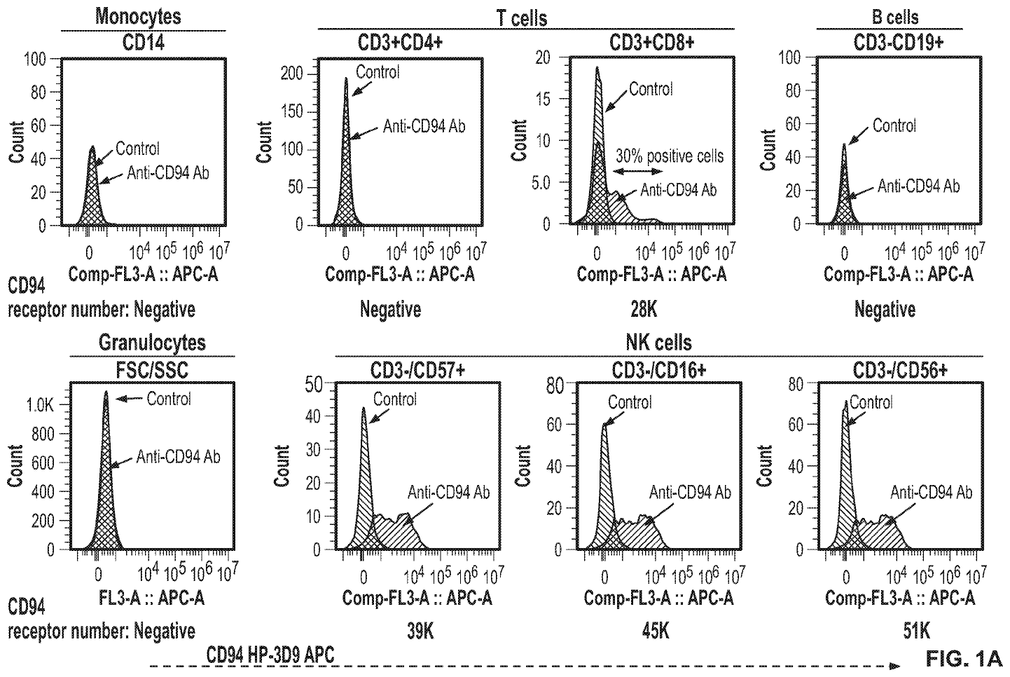

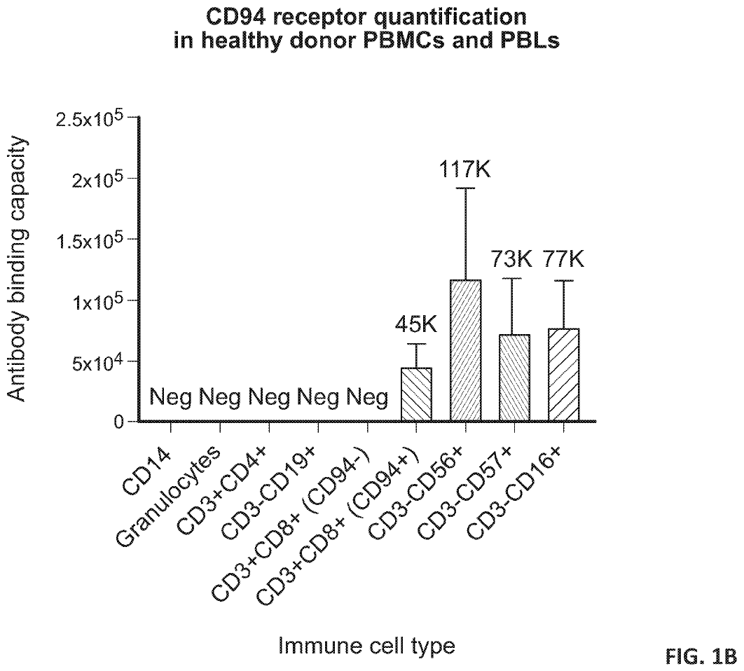

Results of flow cytometry analysis of CD94 in a representative healthy donor PBL sample are provided in FIG. 1A. CD94 was highly expressed on NK cells, with CD94 receptor numbers ranging from about 40,000 to about 50,000 per cell. CD94 was not detected on CD14+ monocytes, granulocytes (FSC/SSC), CD3+CD4+ T-cells and CD3-CD19+ B cells. CD3+CD8+ T cells expressed CD94 in the range of 15-40%. Overall, these results showed that CD94 was selectively expressed on all NK cells, a subset of CD8+ T cells, and was not detected on other cells in a representative healthy donor PBL sample.

FIG. 1B shows an analysis of cell surface CD94 receptor density to quantify CD94 expression on immune cells in samples from six healthy donor PBMC and PBL samples. Specifically, CD94 expression was assessed in six healthy donor PBMC samples using anti-CD94 mAb clone HP-3D9 in CD14+, CD3+CD4+, CD3-CD19+, CD3+CD8+CD94-, CD3+CD8+CD94+, CD3-CD56+, CD3-CD57+, and CD3-CD16+ cells. In addition, CD94 expression on granulocytes was assessed using PBL samples from two healthy donors. CD94 receptor expression was abundant on NK cells, ranging from about 70,000 to about 120,000 receptors/cell (average=117,200). CD94 expression was below 4000 receptors/cell on monocytes (CD14+), granulocytes, T-Cells (CD3+CD4+, CD3+CD8+) and B-Cells (CD3-CD19+). The majority of CD3+CD8+ T cells (60-85%) were CD94-negative. Overall, these results showed that CD94 receptor density was high on all NK cells, low on a subset of CD8+ cells, and not detected in other cell populations in healthy donor PBMC and PBL samples.

T-LGLL Patients

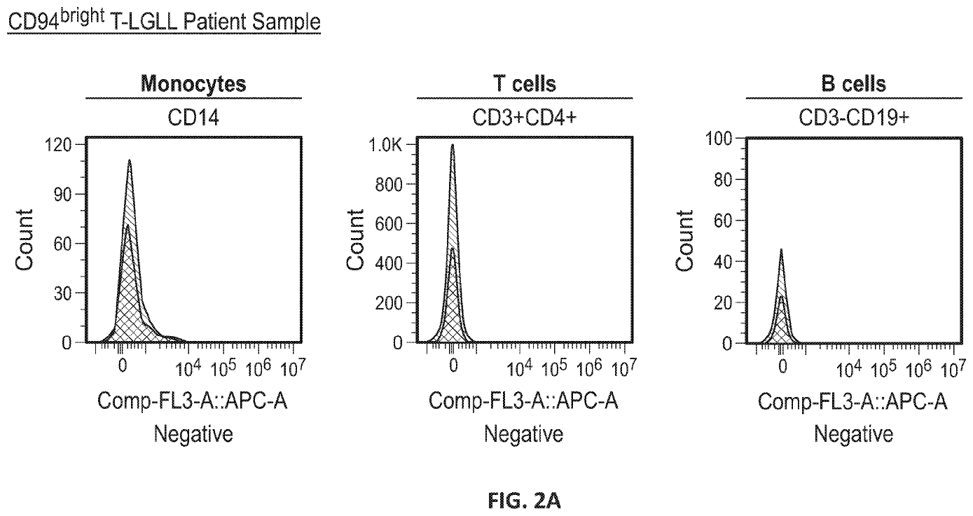

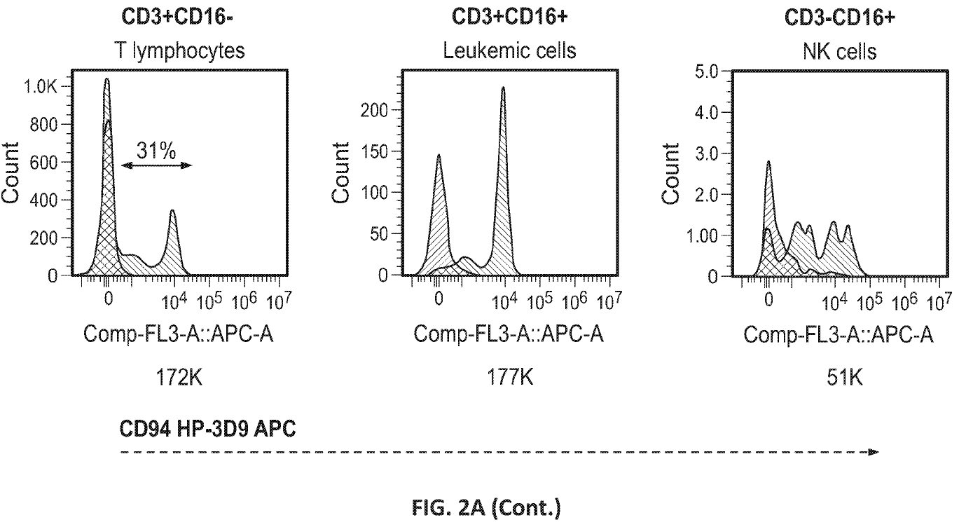

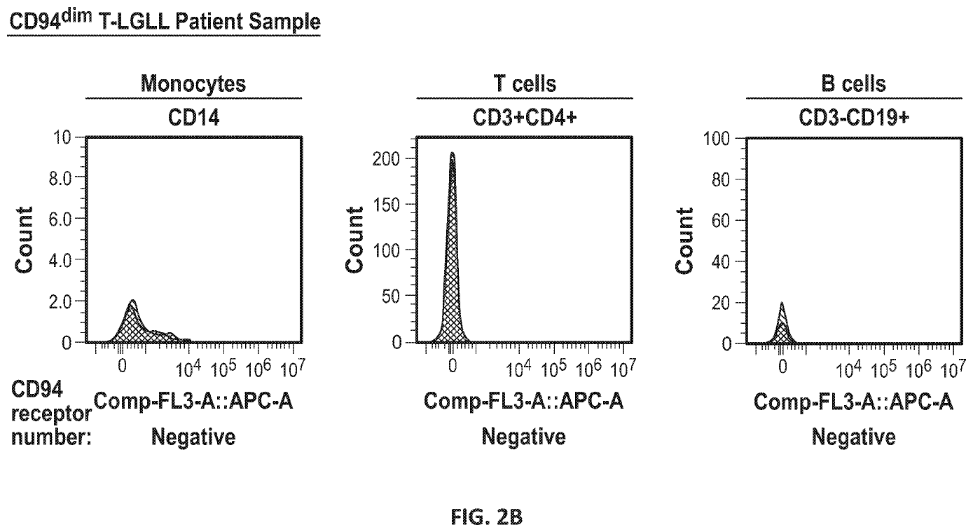

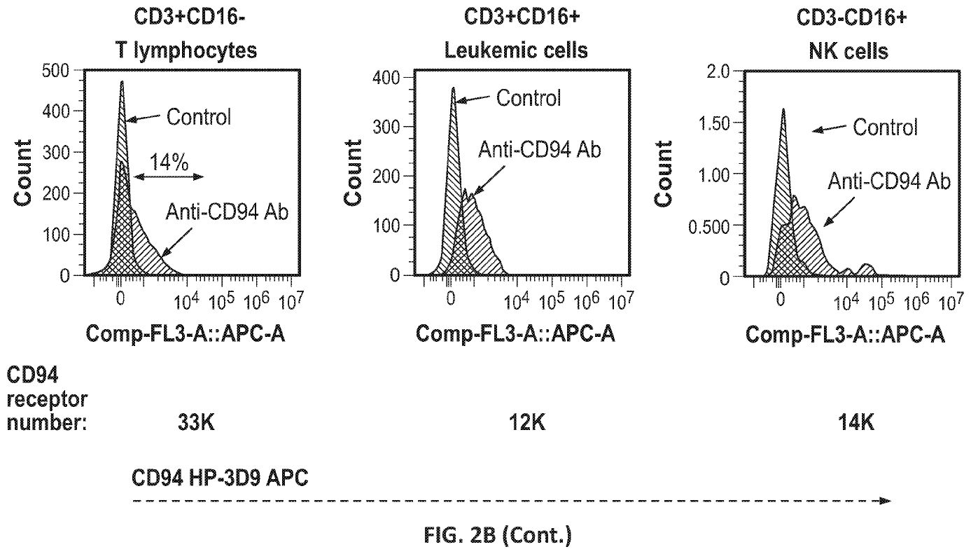

Blood samples from T-LGLL patients were analyzed to determine the number of CD94 receptors on CD14+ monocytes, CD3+CD4+ T cells, CD3-CD19+ B cells, CD3+CD16− T lymphocytes, CD3+CD16+ leukemic cells, and CD3-CD16+ NK cells. CD3+CD16+ leukemic cells represented >55% of lymphocytes in these patient PBMC samples, in comparison to <10% in PBMC samples from healthy individuals. FIG. 2A provides an analysis of CD94 expression and receptor quantification in cells from a CD94^{bright} T-LGLL patient sample, while FIG. 2B shows CD94 expression and receptor quantification in cells from a CD94^{dim} T-LGLL patient sample.

As shown in FIGS. 2A-2B, CD94 was expressed on CD3+CD16+ leukemic cells and a subset of CD3+CD16− T lymphocytes and CD3-CD16+ NK cells. Expression of CD94 on leukemic cells varied between the CD94^{bright} and CD94^{dim} patient samples. As shown in FIG. 2A, in the CD94^{bright} sample, CD3+CD16+ leukemic cells showed >170,000 CD94 receptors and an MFI of 10,000. The high expression of CD94 on CD3+CD16+ leukemic cells in the CD94^{bright} sample suggested that these cells would be completely depleted by ADCC when bound by an anti-CD94 antibody. As shown in FIG. 2B, in the CD94^{dim} sample, CD3+CD16+ leukemic cells showed about 12,000 CD94 receptors and an MFI of 1,000. This discovery was surprising, as others have previously reported that a subset of T-LGLL patients, including patients with immune cell marker profiles similar to the CD94^{dim} patient sample in FIG. 2B, are negative for CD94 expression (see, e.g., Barila (2019) Leukemia). C94 expression on T-LGLL patient monocytes (CD14+), CD3+CD4+ T cells and CD3-CD19+ B cells was not detected in either the CD94^{dim} or the CD94^{bright} patient samples. Overall, these results showed that CD94 was expressed on leukemic cells, NK cells, a subset of CD3+CD16− T cells, and was not detected on other cells in PBMCs from T-LGLL patients. This analysis is thought to represent the first determination of CD94 receptor number on T-LGLL cells. The discovery of CD94 expression on CD3+CD16+ leukemic cells in the CD94^{dim} patient sample suggested that 12,000 receptors expressed on leukemic cells would be sufficient to completely deplete these cells by ADCC when bound by an anti-CD94 antibody.

CLPD-NK Patients

Flow cytometry analysis of CD94 expression and receptor quantification was also carried out in a blood sample from a patient with chronic lymphoproliferative disorder of NK cells (CLPD-NK). CD3-CD16+ leukemic cells represented 70% of lymphocytes in this patient PBMC sample, compared to 5-10% in PBMC samples from healthy individuals.

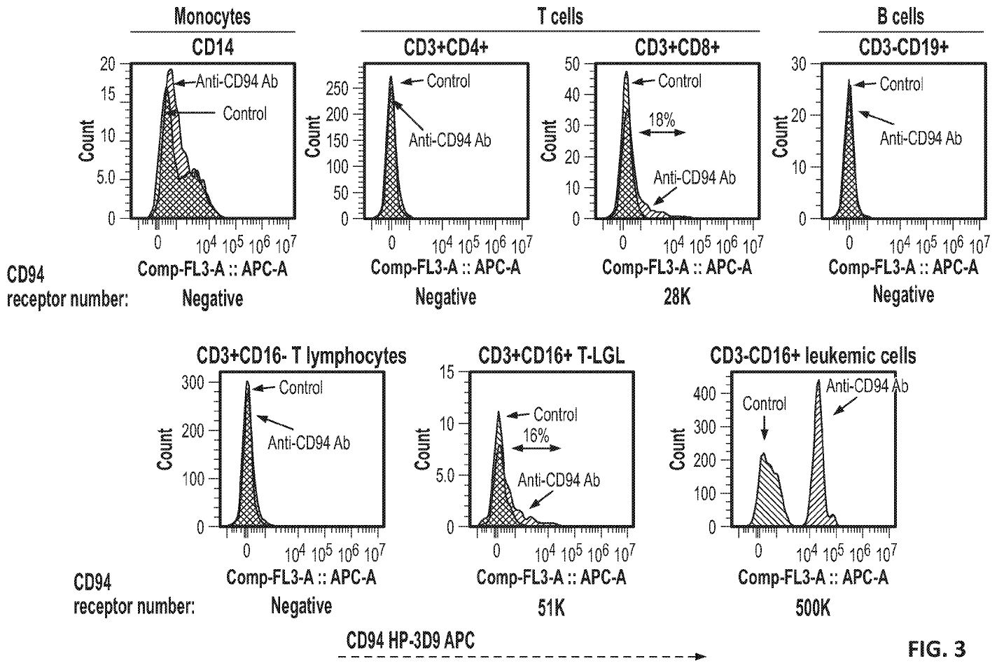

As shown in FIG. 3, CD94 was expressed on CD3-CD16+ NK leukemic cells with a receptor density of 500,000 per cell. CD94 expression was not detected on CLPD-NK patient monocytes (CD14+), CD4+ T-cells (CD3+CD4+), and B cells (CD3-CD19+). CD94 was expressed on a small percentage of CD3+CD8+ T cells (18%). Overall, these results showed that CD94 expression was high on leukemic CLPD-NK cells, low in CD3+CD8+ T-cells, and not detected on other PBMCs from a CLPD-NK patient. This analysis is thought to represent the first determination of CD94 receptor number on CLPD-NK cells. The very high expression of CD94 on leukemic cells suggested that these cells would be completely depleted by ADCC when bound by an anti-CD94 antibody.

### Example 2

### Analysis of NKG2A Expression and the Effects of Anti-NKG2A Antibodies on ADCC Activity in Immune Cells from Healthy Donors and from Patients with Chronic Lymphoproliferative Disorder of NK Cells (CLPD-NK)

This Example describes the results of experiments to determine the level of NKG2A receptor expression on immune cells obtained from healthy donors and from patients with CLPD-NK. This Example also shows results of experiments that measured the effect of anti-NKG2A antibodies on antibody-dependent cellular cytotoxicity (ADCC).

### Materials and Methods

Antibody-Dependent Cellular Cytotoxicity Assay

Approximately 1×10⁵-2×10⁵ fresh or frozen PBMCs were plated in tissue culture-treated 96-well U bottom plates in RPMI with 10% low IgG FBS. The cells were incubated overnight in 10-fold dilutions of human IgG1 isotype control antibody, NKG2A Z199 fucosylated antibody, or NKG2A Z199 non-fucosylated antibody, with antibody concentrations ranging from 10¹-10⁻⁶ μg/ml. The cells were stained with CD3, CD56 and CD16 to identify the remaining NK cells (e.g., as described in Example 1). A minimum of 10,000 events were collected on the flow cytometer in the lymphocyte population. The percent NK/leukemic cells remaining was calculated by normalizing the absolute count by the cell numbers in the isotype treated conditions. The EC50 was determined via GraphPad Prism.

Results

Healthy Donor

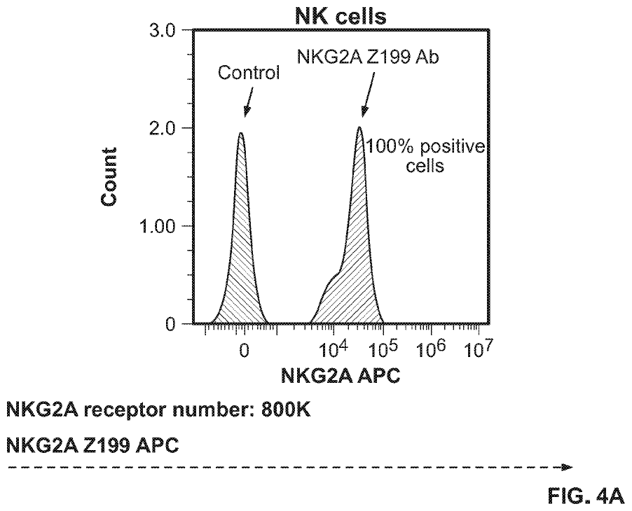

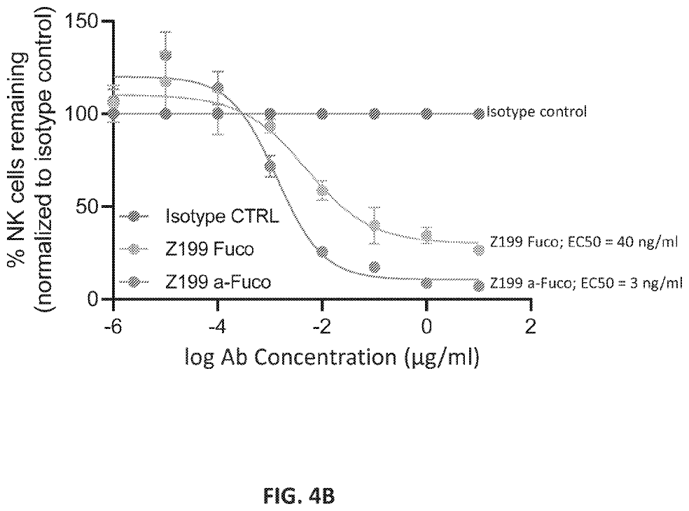

Blood from a healthy donor was analyzed to determine the number of NKG2A receptors on CD56^{bright} NK cells. As shown in FIG. 4A, the NKG2A receptor number on CD3-CD56+ NK cells was 800,000.

To determine whether NK cells can mediate ADCC against NK cells, an ADCC assay was performed in freshly isolated PBMCs from a healthy donor using Z199 fucosylated and non-fucosylated anti-NKG2A antibodies. As shown in FIG. 4B, NK cells were depleted in a dose-dependent manner, with an EC50 of 40 ng/ml and 3 ng/ml for the fucosylated and non-fucosylated Z199 antibody, respectively. Overall, these results showed that the Z199 NKG2A antibody selectively reduced healthy donor NK cells in a dose dependent manner, and that the non-fucosylated antibody was about 13 times more potent than the fucosylated antibody.

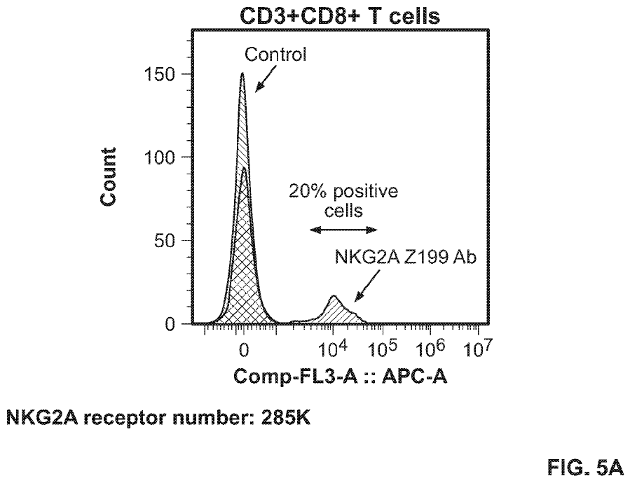

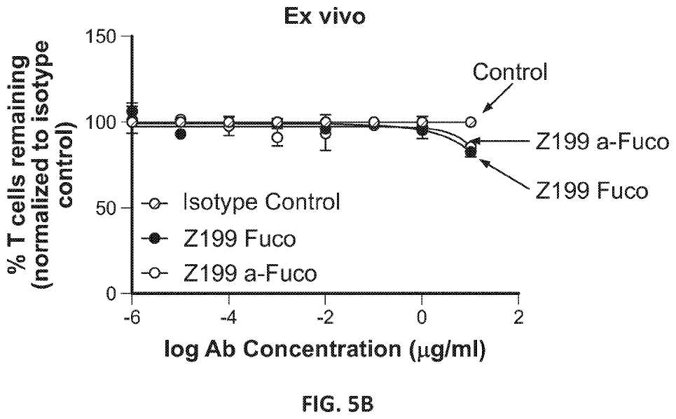

NKG2A expression on T cells from healthy donor PBMC samples was also analyzed. As shown in FIG. 5A, 20% of CD3+CD8+ T cells expressed NKG2A, with a receptor number of 285,000. To determine whether NKG2A-negative cells were resistant to anti-NKG2A Z199 antibody-mediated ADCC killing, an ADCC assay was performed using fresh PBMCs from a healthy donor. The cells were incubated overnight with fucosylated and non-fucosylated IgG1 isotype control and anti-NKG22A Z199 antibodies. As shown in FIG. 5B, the majority of NKG2A-negative CD3+CD8+ T cells were not depleted at all with the tested concentrations of the Z199 antibody. Overall, these results showed that the Z199 NKG2A antibody did not deplete NKG2A-negative T-cells from a healthy donor.

CLPD-NK Patient

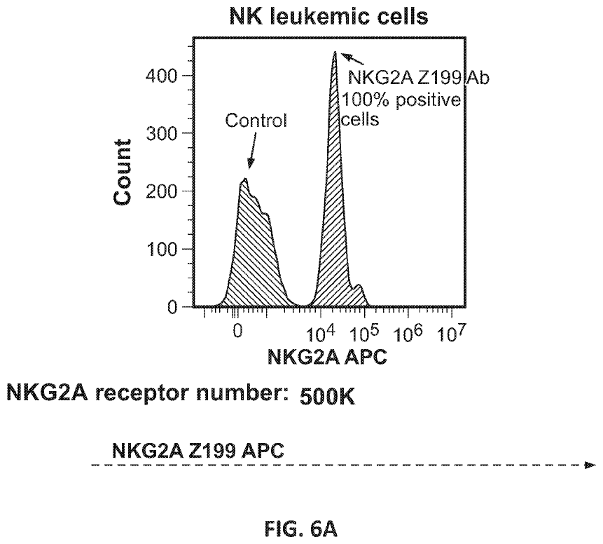

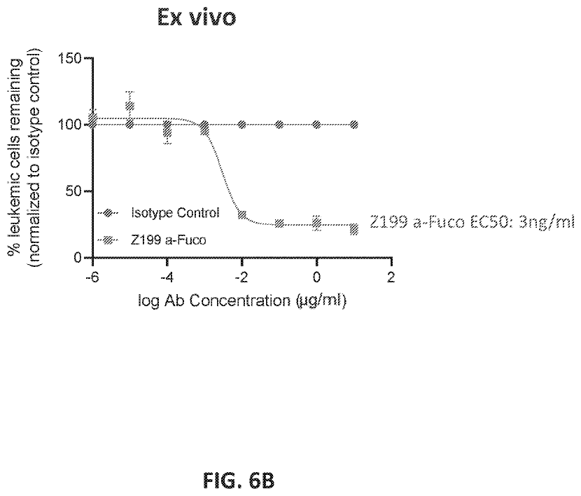

Blood from a CLPD-NK patient was analyzed to determine the number of NKG2A receptors on CD3-CD16+ NK leukemic cells. As shown in FIG. 6A, 100% of CD3-CD16+ NK leukemic cells expressed NKG2A, with an NKG2A receptor number of 500,000.

To determine whether NK leukemic cells can mediate ADCC against NK leukemic cells, an ADCC assay was performed using cells from a previously frozen CLPD-NK patient sample with non-fucosylated IgG1 isotype control and anti-NKG2A Z199 antibodies. As shown in FIG. 6B, NK cells were depleted in a dose-dependent manner with an EC50 of 3ng/ml. Since NK leukemic cells were the only cells with cytotoxic activity in this patient sample (as evidenced by expression of CD16), the observed depletion of leukemic cells suggested that the NK leukemic cells mediated ADCC against the same cell type. Overall, these results showed that non-fucosylated anti-NKG2A Z199 antibody effectively depleted NK leukemic cells (CD3-CD16+).

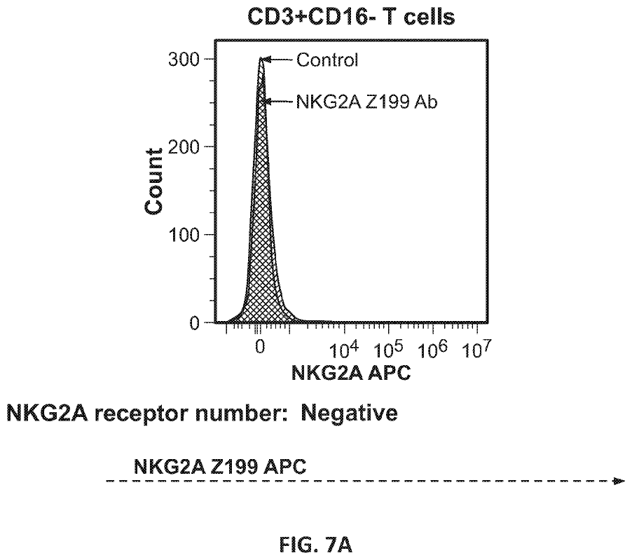

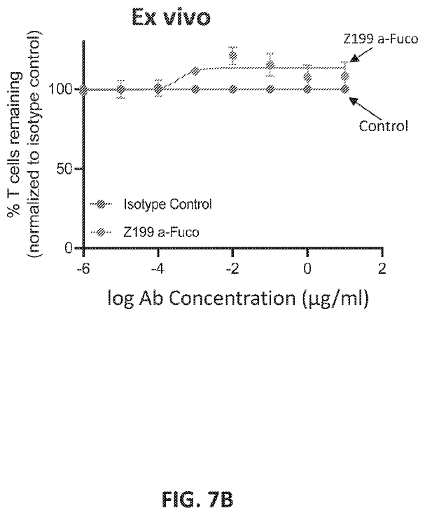

Blood from a CLPD-NK patient was also analyzed to determine the number of NKG2A receptors on CD3+CD16− T cells. As shown in FIG. 7A, CD3+CD16− T cells were negative for NKG2A expression. To determine whether NKG2A-negative cells were resistant to anti-NKG2A Z199 antibody-mediated ADCC killing, an ADCC assay was performed using a previously frozen CLPD-NK patient sample with non-fucosylated IgG1 isotype control and anti-NKG2A Z199 antibodies. As shown in FIG. 7B, NKG2A-negative CD3+CD16− T cells were not depleted at all with the tested concentrations of the anti-NKG2A Z199 antibody. These results showed that the non-fucosylated anti-NKG2A Z199 antibody did not deplete NKG2A-negative T-cells from a CLPD-NK patient.

### Example 3

### Analysis of NKG2A and CD94 Expression on Liver-Derived Cells

This Example describes the results of experiments to determine the level of CD94 and NKG2A receptor expression on liver-derived immune cells obtained from healthy donors.

Materials and Methods

Single and live liver-derived cells (CD45−) and lymphocyte populations (CD45/CD4/CD8/CD19/CD56+) were analyzed by flow cytometry as described in Example 1.

Results

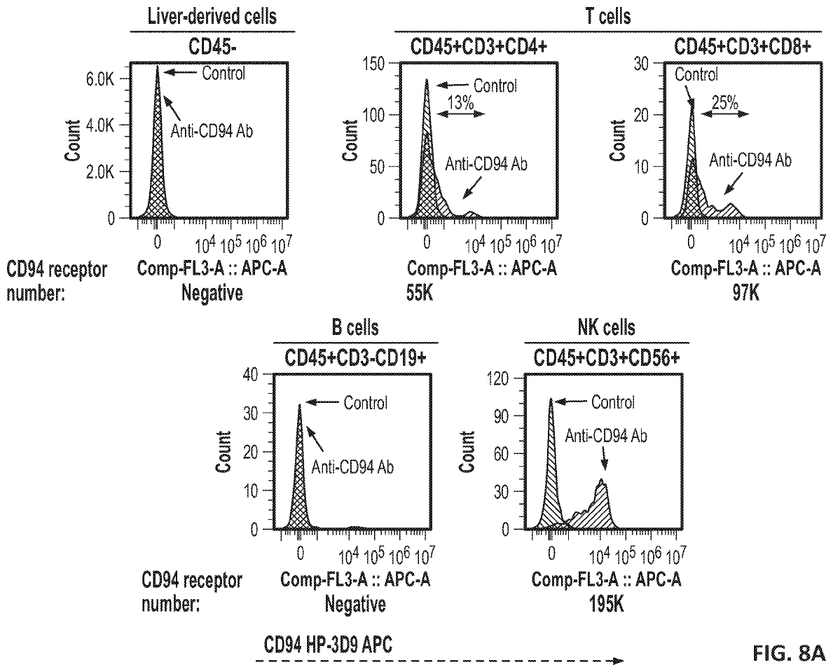

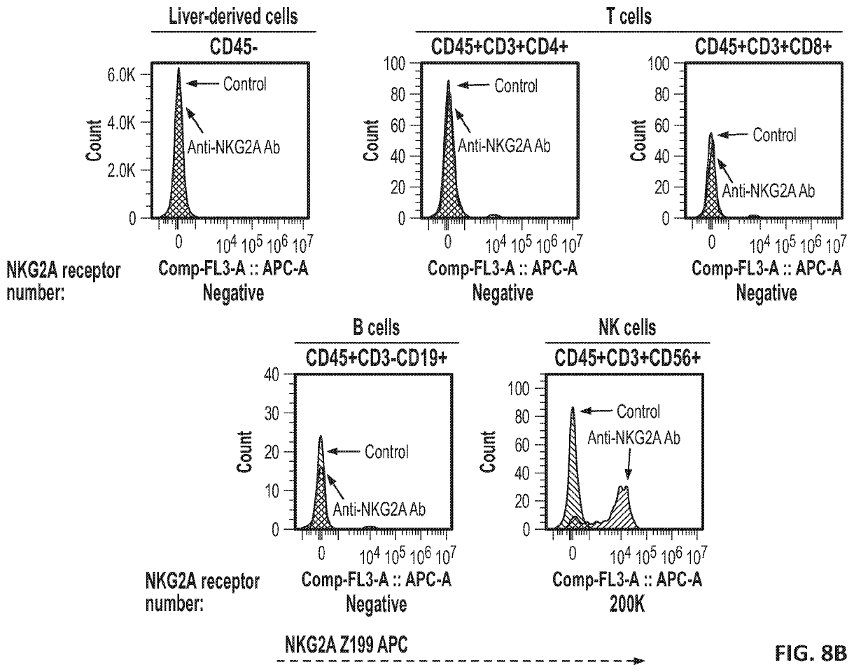

Single and live liver-derived cells (CD45−) and lymphocyte populations (CD45/CD4/CD8/CD19/CD56+) were examined to screen for CD94 and NKG2A expression. As shown in FIG. 8A, CD94 was highly expressed on NK cells of a normal liver sample, with approximately 200,000 CD94 receptors per cell. CD94 expression was also present on a subset of T-cells (CD45+CD3+CD4+/CD8+). CD94 expression was not detected on epithelial cells (CD45−) and B cells (CD45+CD3-CD19+). As shown in FIG. 8B, NKG2A was only detected on NK cells, with a receptor number of 200,000. Overall, these results showed that CD94 and NKG2A were expressed on NK cells in a normal liver sample. CD94 and NKG2A expression was also detected at low levels on T cells in a normal liver sample.

### Example 4

### Analysis of Antibody-Dependent Cellular Cytotoxicity (ADCC) Mediated by Anti-NKG2A Antibody

To determine whether T leukemic cells can mediate ADCC against T leukemic cells, an ADCC assay was performed using cells from a previously frozen T-LGLL patient sample (PBMCs) with non-fucosylated IgG1 isotype control and Z199 antibodies.

Cells were treated with isotype and non-fucosylated Z199 antibody overnight with five concentrations ranging from 0 to lug/ml. The Y-axis is displayed as the number of leukemic cells (CD3+CD16+) remaining in Z199 and human IgG1 isotype treated conditions.

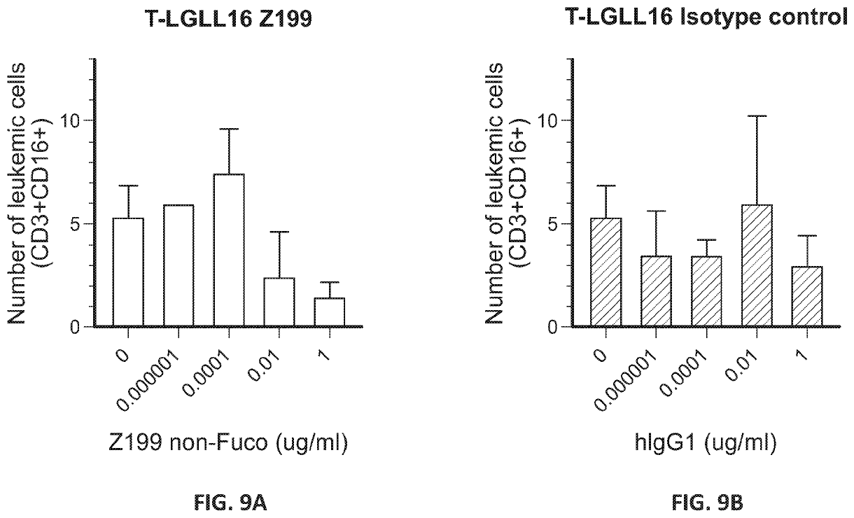

As shown in FIGS. 9A & 9B, T-LGLL cells were depleted in a dose-dependent manner by the Z199 antibody, but not isotype control. Since the T-LGL leukemic cells were the only cells with cytotoxic activity (as evidenced by expression of CD16) in this patient sample, the depletion of leukemic cells suggests that the leukemic cells were mediating ADCC against the same cell type.

These results demonstrate that the non-fucosylated, anti-NKG2A antibody Z199 effectively depletes T leukemic cells.

### Example 5: Effect of IL-2 on CD94 Expression

CD94 expression was measured over time in normal NK cells cultured with IL-2.

NK cells purified from healthy donor PBMCs were cultured in IL-2 (50 ng/ml) from day 0 to 4. CD94 expression, displayed as median fluorescence intensity by flow cytometry, was determined by comparing to fluorescence minus one (FMO) and isotype control.

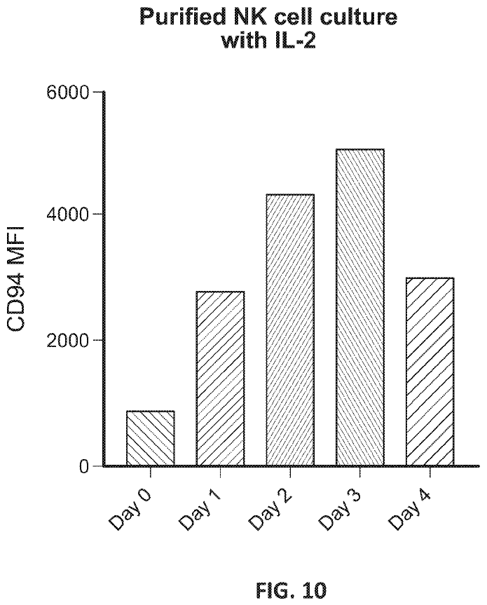

As shown in FIG. 10, CD94 expression increased over time during culture with IL-2 treatment. These results demonstrate that CD94 expression on NK cells was upregulated in the presence of IL-2.

Although the present disclosure has been described in some detail by way of illustration and example for purposes of clarity of understanding, the descriptions and examples should not be construed as limiting the scope of the present disclosure. The disclosures of all patent and scientific literature cited herein are expressly incorporated in the entirety by reference.

What is claimed is:

1. A method for treating a disease or disorder in a subject, comprising administering to the subject an effective amount of an antibody that specifically binds to a cell surface protein selected from the group consisting of human CD94, human CD57, and human NKG2A, wherein the antibody comprises a human immunoglobulin Fc region comprising enhanced ADCC activity compared to a wild type IgG1 Fc region, and wherein the disease or disorder is selected from the group consisting of chronic lymphoproliferative disorder of NK cells (CLPD-NK), LGL leukemia, Felty's syndrome, rheumatoid arthritis, aggressive NK leukemia, inclusion body myositis, and inflammatory bowel disease.

2. The method of claim 1, wherein administration of the antibody results in a reduction in the number of peripheral blood LGL or NK cells in the subject.

3. A method for reducing the number of peripheral blood LGL and/or NK cells in a subject, comprising administering to the subject an effective amount of an antibody that specifically binds to a cell surface protein selected from the group consisting of human CD94, human CD57, and human NKG2A, wherein the antibody comprises a human immunoglobulin Fc region comprising enhanced ADCC activity compared to a wild type IgG1 Fc region, and wherein the subject has a disease or disorder selected from the group consisting of LGL leukemia, Felty's syndrome, rheumatoid arthritis, aggressive NK leukemia, inclusion body myositis, and inflammatory bowel disease.

4. A method for inducing ADCC activity in a subject, comprising administering to the subject an effective amount of an antibody that specifically binds to a cell surface protein selected from the group consisting of human CD94, human CD57, and human NKG2A, wherein the antibody comprises a human immunoglobulin Fc region comprising enhanced ADCC activity compared to a wild type IgG1 Fc region, wherein the subject has a disease or disorder selected from the group consisting of chronic lymphoproliferative disorder of NK cells (CLPD-NK), LGL leukemia, Felty's syndrome, rheumatoid arthritis, aggressive NK leukemia, inclusion body myositis, and inflammatory bowel disease, and wherein administration of the antibody to the subject results in a reduction in the number of peripheral blood LGL and/or NK cells in the subject.

5. The method of any one of claims 2-4, wherein at least about 2,000 receptors per cell of the cell surface protein are expressed on the surface of the peripheral blood LGL and/or NK cells in the subject.

6. The method of any one of claims 2-5, wherein the reduction in the number of peripheral blood LGL or NK cells in the subject comprises a reduction of at least about 25% compared to the number of peripheral blood NK cells in the human subject prior to administration of the antibody.

7. The method of any one of claims 2-6, wherein the reduction in the number of peripheral blood LGL and/or NK cells in the subject occurs within the first 24 hours after administration of the antibody to the subject.

8. The method of any one of claims 2-7, wherein the number of peripheral blood LGL and/or NK cells in the subject is reduced to below the limit for clinical diagnosis of the disease or disorder.

9. The method of claim 8, wherein the reduction in the number of peripheral blood LGL and/or NK cells in the subject to below the limit for clinical diagnosis of the disease or disorder is present in the subject for at least about 1 week after administration of the antibody to the subject.

10. The method of any one of claims 2-9, wherein the number of peripheral blood LGL and/or NK cells in the subject is reduced to below the limit of detection for the peripheral blood LGL and/or NK cells in the subject.

11. The method of claim 10, wherein the reduction in the number of peripheral blood LGL and/or NK cells in the subject to below the limit of detection for the peripheral blood LGL and/or NK cells is present in the subject for at least about 1 week after administration of the antibody to the subject.

12. The method of any one of claims 2-11, wherein the reduction in the number of peripheral blood LGL and/or NK cells in the subject is reversible.

13. The method of any one of claims 1-12, wherein administration of the antibody to the subject results in a reduction in the number of peripheral blood NK cells in the subject.

14. The method of claim 13, wherein the NK cells in the subject are CD3 negative and CD56 positive, CD3 negative and CD16 positive, CD3 negative and CD57 positive, CD3 negative and CD94 positive, or CD3 negative and NKG2A positive.

15. The method of claim 13 or claim 14, wherein the antibody has an EC50 of between about 3 ng/ml and about 40 ng/ml.

16. The method of any one of claims 1-15, wherein administration of the antibody to the subject does not result in a reduction of T cells in the subject.

17. The method of claim 16, wherein the T cells in the subject are CD3 positive and CD4 positive or CD3 positive and CD16 negative.

18. The method of any one of claims 1-17, wherein the subject is a human.

19. The method of any of claims 1-18, wherein administration of the antibody to the subject does not result in tumor lysis syndrome in the subject.

20. The method of any of claims 1-19, wherein the antibody comprises a human IgG1 Fc region that is non-fucosylated.

21. The method of any one of claims 1-20, wherein the antibody binds to a human cellular Fc gamma receptor IIIA to a greater extent than an antibody comprising a wild type human IgG1 Fc region.

22. The method of claim 21, wherein the human cellular Fc gamma receptor IIIA comprises the sequence of SEQ ID NO: 8 or 9.

23. The method of any one of claims 1-22, wherein the antibody:
(a) specifically binds to human CD94, wherein the antibody does not bind to the same epitope on human CD94 as anti-CD94 antibody clones HP-3D9, DX22, 131412, or 12K45;
(b) specifically binds to human CD57, wherein the antibody does not bind to the same epitope on human CD57 as anti-CD57 antibody clone NK-1; or
(c) specifically binds to human NKG2A, wherein the antibody does not bind to the same epitope on human NKG2A as anti-NKG2A antibody clone Z199.

24. The method of any one of claims 1-22, wherein the antibody:
(a) specifically binds to human CD94, wherein the antibody binds to human CD94 with a greater affinity than anti-CD94 antibody clones HP-3D9, DX22, 131412, and 12K45;
(b) specifically binds to human CD57, wherein the antibody binds to human CD57 with greater affinity than anti-CD57 antibody clone NK-1; or
(c) specifically binds to human NKG2A, wherein the antibody binds to human NKG2A with a greater affinity than anti-NKG2A antibody clone Z199.

25. The method of any one of claims 1-24, wherein the disease or disorder is Felty's syndrome, and wherein administration of the antibody to the subject results in a reduction of one or more Felty's syndrome symptoms in the subject.

26. The method of any one of claims 1-24, wherein the disease or disorder is inclusion body myositis, and wherein administration of the antibody to the subject results in a reduction of one or more inclusion body myositis symptoms in the subject.

27. The method of any one of claims 1-24, wherein the disease or disorder is aggressive NK leukemia, and wherein administration of the antibody to the subject results in a reduction of one or more aggressive NK leukemia symptoms in the subject.

28. The method of any one of claims 1-24, wherein the disease or disorder is rheumatoid arthritis, and wherein administration of the antibody to the subject results in a reduction of one or more rheumatoid arthritis symptoms in the subject.

29. The method of any one of claims 1-24, wherein the disease or disorder is LGL leukemia, and wherein administration of the antibody to the subject results in a reduction of one or more LGL leukemia symptoms in the subject.

30. The method of any one of claims 1-24, wherein the disease or disorder is CLPD-NK, and wherein administration of the antibody to the subject results in a reduction of one or more CLPD-NK symptoms in the subject.

31. A method for treating CLPD-NK in a human subject in need thereof, comprising administering to the human subject an effective amount of an antibody, wherein the antibody specifically binds to human NKG2A, and wherein the antibody comprises a human immunoglobulin Fc region comprising enhanced ADCC activity compared to a wild type IgG1 Fc region.

32. The method of claim 31, wherein the antibody does not bind to the same epitope on human NKG2A as anti-NKG2A antibody clone Z199.

33. The method of claim 31 or claim 32, wherein the antibody binds to human NKG2A with a greater affinity than anti-NKG2A antibody clone Z199.

34. A method for treating CLPD-NK in a human subject in need thereof, comprising administering to the human subject an effective amount of an antibody, wherein the antibody specifically binds to human CD94, and wherein the antibody comprises a human immunoglobulin Fc region comprising enhanced ADCC activity compared to a wild type IgG1 Fc region.

35. The method of claim 34, wherein the antibody does not bind to the same epitope on human CD94 as anti-CD94 antibody clones HP-3D9, DX22, 131412, or 12K45.

36. The method of claim 34 or claim 35, wherein the antibody binds to human CD94 with a greater affinity than anti-CD94 antibody clones HP-3D9, DX22, 131412, and 12K45.

37. The method of any one of claims 31-36, wherein administration of the antibody to the human subject results in a reduction in the number of peripheral blood LGL or NK cells in the human subject of at least about 25% compared to the number of peripheral blood NK cells in the human subject prior to administration of the antibody.

38. The method of any one of claims 31-37, wherein the NK cells in the human subject are CD3 negative and CD56 positive, CD3 negative and CD16 positive, CD3 negative and CD57 positive, CD3 negative and CD94 positive, or CD3 negative and NKG2A positive.

39. The method of any one of claims 31-38, wherein administration of the antibody to the human subject does not result in a reduction of T cells in the human

40. The method of claim 39, wherein the T cells in the human subject are CD3 positive and CD4 positive or CD3 positive and CD16 negative.

41. The method of any of claims 31-40, wherein administration of the antibody to the human subject does not result in tumor lysis syndrome in the human

42. The method of any of claims 31-41, wherein the antibody comprises a human IgG1 Fc region that is non-fucosylated.

43. The method of any one of claims 31-42, wherein the antibody binds to a human cellular Fc gamma receptor IIIA to a greater extent than an antibody comprising a wild type human IgG1 Fc region.

44. The method of claim 43, wherein the human cellular Fc gamma receptor IIIA comprises the sequence of SEQ ID NO: 8 or 9.

45. The method of any one of claims 31-44, wherein administration of the antibody to the human subject results in an improvement of one or more CLPD-NK symptoms in the human.


SEQUENCE LISTING (9 sequences)

SEQ ID NO: 1 - protein, 179 aa, Homo sapiens
MAVFKTTLWRLISGTLGIICLSLMSTLGILLKNSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 2 - protein, 180 aa, Homo sapiens
MAVFKTTLWRLISGTLGIICLSLMSTLGILLKNSFTKLSIEPAFTPGPNIELQKDSDCCSCQEKWVGYRCNCYFISSEQKTWNESRHLCASQKSSLLQLQNTDELQDFMSSSQQFYWIGLSYSEEHTAWLWENGSALSQYLFPSFETFNTKNCIAYNPNGNALDESCEDKNRYICKQQLI

SEQ ID NO: 3 - protein, 208 aa, Homo sapiens
MAAFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLISYSEEHTAWLWENGSALSQYLFPSFETFNTKNCIAYNPNGNALDESCEDKNRYICKQQLI

SEQ ID NO: 4 - protein, 233 aa, Homo sapiens
MDNQGVIYSDLNLPPNPKRQQRKPKGNKNSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 5 - protein, 215 aa, Homo sapiens
MDNQGVIYSDLNLPPNPKRQQRKPKGNKNSILATEQEITYAELNLQKASQDFQGNDKTYHCKDLPSAPEKLIVGILGIICLILMASVVTIVVIPSRHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 6 - protein, 195 aa, Homo sapiens
MTDSVIYSMLELPTATQAQNDYGPQQKSSSSRPSCSCLVAIALGLLTAVLLSVLLYQWILCQGSNYSTCASCPSCPDRWMKYGNHCYYFSVEEKDWNSSLEFCLARDSHLLVITDNQEMSLLQVFLSEAFCWIGLRNNSGWRWEDGSPLNFSRISSNSFVQTCGAINKNGLQASSCEVPLHWVCKKCPFADQALF

SEQ ID NO: 7 - protein, 189 aa, Homo sapiens
MTDSVIYSMLELPTATQAQNDYGPQQKSSSSRPSCSCLVAIALGLLTAVLLSVLLYQWILCQGSNYSTCASCPSCPDRWMKYGNHCYYFSVEEKDWNSSLEFCLARDSHLLVITDNQEMSLLQVFLSEAFCWIGLRNNSGWRWEDGSPLNFSRISSNSFVQTCGAINKNGLQASSCEVPLHWVCKKVRL

SEQ ID NO: 8 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 9 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK